(19) United States
(12) Patent Application Publication
(10) Pub. No.: US 2023/0220475 A1
(43) Pub. Date: Jul. 13, 2023

(54) COMPOSITIONS AND METHODS FOR DNA METHYLATION ANALYSIS

(71) Applicant: President and Fellows of Harvard College, Cambridge, MA (US)

(72) Inventors: Patrick Thomas Griffin, Cambridge, MA (US); David A. Sinclair, Cambridge, MA (US)

(21) Appl. No.: 18/008,989

(22) PCT Filed: Jun. 11, 2021
(86) PCT No.: PCT/US2021/037069

§ 371 Date: Dec. 8, 2022

Related U.S. Application Data
(60) Provisional application No. 63/038,157, filed on Jun. 12, 2020.

(51) Int. Cl. C12Q 1/6883

(52) U.S. Cl. CPC C12Q 1/6883; C12Q 2600/154; C12Q 2600/16; C12Q 1/6806

(57) ABSTRACT

The present invention provides methods, compositions and kits for assembling an enzyme-deoxyribonucleic acid (DNA) complex for use in preparing a double stranded DNA molecule comprising one or more loci of interest for determining the methylation status of the one or more loci of interest therein.

### CROSS-REFERENCE TO RELATED APPLICATIONS

This application claims the benefit of priority to U.S. Provisional Application No. 63/038,157, filed on Jun. 12, 2020, the entire contents of which are expressly incorporated herein by reference.

### GOVERNMENT SUPPORT

This invention was made with Government support under AG019719 awarded by the National Institutes of Health. The Government has certain rights in the invention.

### TECHNICAL FIELD

The present invention relates to compositions and methods for determining the cytosine methylation status of one or more loci of interest contained within a double stranded DNA molecule.

### BACKGROUND OF THE INVENTION

An epigenetic mark called DNA cytosine methylation (DNAm) is a conserved epigenetic modification that influences expression of genes. The DNAm status of a genome can be used as a mark to determine, inter alia, the status of a cancer, the biological age of a blood or tissue sample, and for forensics. Even though the cost of DNA sequencing has fallen dramatically, the cost of reading DNAm marks in a genome is still too high for it to be used widely and on millions of samples. Indeed, each analysis can cost between $200-600. If the cost of the tests could be reduced 10- to 100-fold, it would be used routinely in medicine and research.

Accordingly, there is a need in the art for inexpensive compositions and facile methods to determine the DNAm status of a genome.

### SUMMARY OF THE INVENTION

The present invention is based upon, at least partly, the discovery that the methylation status of one or more loci of interest on a deoxyribonucleic acid (DNA) molecule can be determined with high level of fidelity using a method that includes the use of cytosine free oligonucleotides as adaptors. Accordingly, the present invention provides compositions, kits, and methods for determining the methylation status of one or more loci of interest present in a DNA molecule.

Accordingly, in one aspect, the present invention provides a method for assembling an enzyme-deoxyribonucleic acid (DNA) complex for use in preparing a double stranded DNA molecule comprising one or more loci of interest for determining the methylation status of the one or more loci of interest therein. The method includes contacting an enzyme with a first partially double stranded oligonucleotide comprising a first adaptor single stranded oligonucleotide and a first barcode single stranded oligonucleotide, wherein the first adaptor oligonucleotide and the first barcode oligonucleotide are operably linked in the order, from 5′ to 3′, the first adaptor-the first barcode, and a second partially double stranded oligonucleotide comprising a second adaptor single stranded oligonucleotide, wherein the enzyme is capable of operably linking the first and the second partially double stranded oligonucleotides to the double stranded DNA molecule comprising one or more loci of interest; wherein the first adaptor and the first barcode do not comprise a cytosine, wherein the second adaptor does not comprise a cytosine or the cytosine thereon is methylated; and wherein the nucleotide sequence of the first adaptor and the second adaptor are different, thereby preparing the enzyme-DNA complex.

In one embodiment, the first partially double stranded oligonucleotide further comprises a first enzyme recognition sequence, wherein the first enzyme recognition sequence is operably linked to the 3′-terminus of the first barcode; and wherein the second partially double stranded oligonucleotide further comprises a second enzyme recognition sequence, wherein the second enzyme recognition sequence is operably linked to the 3′-terminus of the second adaptor. In one embodiment, the first enzyme recognition sequence and the second enzyme recognition sequence comprise the same sequence. In another embodiment, the first partially double stranded oligonucleotide and the second partially double stranded oligonucleotide contact the enzyme concurrently in a same reaction mixture.

In another embodiment, the first enzyme recognition sequence is a first transposon end sequence for a transposon, and wherein the second enzyme recognition sequence is a second transposon end sequence for the transposon. In one embodiment, the first transposon end sequence and the second transposon sequence comprise the same sequence.

In still another embodiment, the enzyme is a transposase, and the enzyme-DNA complex is a transposome. In yet another embodiment, the transposon is transposon 5 (Tn5).

In one embodiment, the enzyme is a hyperactive transposase Tn5.

In another embodiment, the transposon end sequence comprises a hyperactive mosaic end (ME) nucleotide sequence. In still another embodiment, wherein the nucleotide sequence of the sense strand of the ME sequence is at least about 85%, about 90%, about 91%, about 92%, about 93%, about 94%, about 95%, about 96%, about 97%, about 98%, about 99%, or about 100% identical to the entire nucleotide sequence of a nucleotide sequence having the sequence of SEQ ID NO: 1.

In still another embodiment, the first adaptor is between 6 nucleotides and 30 nucleotides in length. In one embodiment, the first adaptor is 6, 7, 8, 9, 10, 11, 12, 13, 14, 15, 16, 17, 18, 19, 20, 21, 22, 23, 24, 25, 26, 27, 28, 29, or 30 nucleotides in length. In yet another embodiment, the first adaptor 14 nucleotides in length. In one embodiment, the first adaptor comprises a nucleotide sequence having at least about 85%, about 90%, about 91%, about 92%, about 93%, about 94%, about 95%, about 96%, about 97%, about 98%, about 99%, or about 100% nucleotide identity to the entire nucleotide sequence of SEQ ID NO: 4, wherein the first adaptor does not comprise a cytosine.

In one embodiment, the second adaptor is between 6 nucleotides and 30 nucleotides in length. In one embodiment, the second adaptor is 6, 7, 8, 9, 10, 11, 12, 13, 14, 15, 16, 17, 18, 19, 20, 21, 22, 23, 24, 25, 26, 27, 28, 29, or 30 nucleotides in length. In another embodiment, the second adaptor is 15 nucleotides in length. In still another embodiment, the second adaptor comprises a nucleotide sequence having at least about 85%, about 90%, about 91%, about 92%, about 93%, about 94%, about 95%, about 96%, about 97%, about 98%, about 99%, or about 100% nucleotide identity to the entire nucleotide sequence of SEQ ID NO: 5, wherein the cytosine on the second adaptor is methylated. In yet another embodiment, the second adaptor comprises a nucleotide sequence having entire nucleotide sequence of SEQ ID NO: 5.

In one embodiment, the second adaptor is between 6 nucleotides and 30 nucleotides in length. In one embodiment, the second adaptor is 6, 7, 8, 9, 10, 11, 12, 13, 14, 15, 16, 17, 18, 19, 20, 21, 22, 23, 24, 25, 26, 27, 28, 29, or 30 nucleotides in length. In another embodiment, the second adaptor is 15 nucleotides in length. In still another embodiment, the second adaptor comprises a nucleotide sequence having at least about 85%, about 90%, about 91%, about 92%, about 93%, about 94%, about 95%, about 96%, about 97%, about 98%, about 99%, or about 100% nucleotide identity to the entire nucleotide sequence of SEQ ID NO: 16, wherein the cytosine on the second adaptor is methylated. In yet another embodiment, the second adaptor comprises a nucleotide sequence having entire nucleotide sequence of SEQ ID NO: 16.

In another embodiment, the first barcode comprises a nucleotide sequence selected from the group consisting of DDDDD and DDDDDD.

In another aspect, the present invention provides method of preparing a double stranded deoxyribonucleic acid (DNA) molecule comprising one or more loci of interest for determining the methylation status of one or more loci of interest therein. The method includes providing a double stranded DNA molecule comprising one or more loci of interest, contacting the double stranded DNA molecule comprising one or more loci of interest with the enzyme-DNA complex prepared according to the method of any one embodiment of any above aspects of the invention.

In still another aspect, the present invention provides a method of preparing a double stranded deoxyribonucleic acid (DNA) molecule comprising one or more loci of interest for determining the methylation status of the one or more loci of interest therein. The method includes providing a double stranded DNA molecule comprising one or more loci of interest, the DNA molecule comprising a first strand and a second strand; operably linking a first partially double stranded oligonucleotide comprising a first adaptor single stranded oligonucleotide and a first barcode single stranded oligonucleotide to the 5′-terminus of the first strand of the double stranded DNA molecule in the order, from 5′ to 3′, the first adaptor-the first barcode-the double strand DNA molecule; and operably linking a second partially double stranded oligonucleotide comprising a second adaptor single stranded oligonucleotide to the 5′-terminus of the second strand of the DNA molecule, wherein the first adaptor and the first barcode do not comprise a cytosine, wherein the second adaptor does not comprise a cytosine or the cytosine thereon is methylated; and wherein the nucleotide sequence of the first adaptor and the second adaptor are different, thereby preparing the double stranded DNA comprising one or more loci of interest for determining the methylation status of the one or more loci of interest therein.

In one embodiment, the first partially double stranded oligonucleotide further comprises a first enzyme recognition sequence, wherein the first enzyme recognition sequence is operably linked to the 3′-terminus of the first barcode and the 5′-terminus of the first strand of the DNA; and wherein the second partially double stranded oligonucleotide further comprises a second enzyme recognition sequence, wherein the second enzyme recognition sequence is operably linked to the 3′-terminus of the second adaptor and the 5′-terminus of the second strand of the DNA. In one embodiment, the first enzyme recognition sequence and the second enzyme recognition sequence comprise the same sequence.

In another embodiment, the first enzyme recognition sequence is a first end sequence for a transposon, and wherein the second enzyme recognition sequence is a second end sequence for the transposon. In one embodiment, the first end sequence and the second end sequence comprise the same sequence. In still another embodiment, the transposon is a hyperactive transposon 5 (Tn5). In yet another embodiment, the end sequence comprises a hyperactive mosaic end (ME) nucleotide sequence. In yet another embodiment, the nucleotide sequence of the sense strand of the ME sequence is at least about 85%, about 90%, about 91%, about 92%, about 93%, about 94%, about 95%, about 96%, about 97%, about 98%, about 99%, or about 100% identical to the entire nucleotide sequence of SEQ ID NO: 1.

In still another embodiment, the method further comprises assembling a transposome, comprising contacting a transposase with the first partially double stranded oligonucleotide and the second partially double stranded oligonucleotide. In one embodiment, the first partially double stranded oligonucleotide and the second partially double stranded oligonucleotide contact the transposase concurrently in a same reaction mix. In yet another embodiment, the method further comprises contacting the transposome with the double stranded DNA molecule comprising one or more loci of interest, wherein the transposome fragments the double stranded DNA molecule comprising one or more loci of interest and operably links the first partially double stranded oligonucleotide and the second partially double stranded oligonucleotide to the double stranded DNA molecule comprising one or more loci of interest.

In one embodiment, the first adaptor is between 6 nucleotides and 30 nucleotides, or between 14 nucleotides and 20 nucleotides in length. In one embodiment, the first adaptor is 6, 7, 8, 9, 10, 11, 12, 13, 14, 15, 16, 17, 18, 19, 20, 21, 22, 23, 24, 25, 26, 27, 28, 29, or 30 nucleotides in length. In another embodiment, the first adaptor is 14 nucleotides in length. In still another embodiment, the first adaptor comprises a nucleotide sequence having at least about 85%, about 90%, about 91%, about 92%, about 93%, about 94%, about 95%, about 96%, about 97%, about 98%, about 99%, or about 100% nucleotide identity to the entire nucleotide sequence of SEQ ID NO: 4, wherein the first adaptor does not comprise a cytosine.

In another embodiment, the second adaptor is between 6 nucleotides and 30 nucleotides in length. In one embodiment, the second adaptor is 6, 7, 8, 9, 10, 11, 12, 13, 14, 15, 16, 17, 18, 19, 20, 21, 22, 23, 24, 25, 26, 27, 28, 29, or 30 nucleotides in length. In still another embodiment, the second adaptor is 15 nucleotides in length. In yet another embodiment, the second adaptor comprises a nucleotide sequence having at least about 85%, about 90%, about 91%, about 92%, about 93%, about 94%, about 95%, about 96%, about 97%, about 98%, about 99%, or about 100% nucleotide identity to the entire nucleotide sequence of SEQ ID NO: 5, wherein the cytosine on the second adaptor is methylated.

In another embodiment, the second adaptor is between 6 nucleotides and 30 nucleotides in length. In one embodiment, the second adaptor is 6, 7, 8, 9, 10, 11, 12, 13, 14, 15, 16, 17, 18, 19, 20, 21, 22, 23, 24, 25, 26, 27, 28, 29, or 30 nucleotides in length. In still another embodiment, the second adaptor is 15 nucleotides in length. In yet another embodiment, the second adaptor comprises a nucleotide sequence having at least about 85%, about 90%, about 91%, about 92%, about 93%, about 94%, about 95%, about 96%, about 97%, about 98%, about 99%, or about 100% nucleotide identity to the entire nucleotide sequence of SEQ ID NO: 16, wherein the cytosine on the second adaptor is methylated.

In still another embodiment, the first barcode comprises a nucleotide sequence selected from the group consisting of DDDDD and DDDDDD.

In one embodiment, the method further comprises repairing the ends of double stranded DNA molecule comprising one or more loci of interest operably linked to the first partially double stranded oligonucleotide and the partially double stranded second oligonucleotide using methylated cytosine, thereby generating an end repaired double stranded DNA comprising one or more loci of interest. In another embodiment, a Klenow, a T4 polymerase, or a mixture thereof is used for the end repairing.

In still another embodiment, the method further comprises enriching the DNA molecule comprising one or more loci of interest following end repairing, thereby generating a enriched DNA molecule comprising one or more loci of interest. In one embodiment, the enriched DNA molecule comprising one or more loci of interest is a single stranded DNA molecule. In yet another embodiment, the enrichment method is an in-solution target enrichment method. In another embodiment, the enrichment comprises in-solution biotinylated RNA bait hybridization.

In one embodiment, the method further comprises converting the unmethylated cytosine in the end repaired double stranded DNA molecule comprising one or more loci of interest or the enriched DNA molecule comprising one or more loci of interest to uracil, thereby generating a cytosine-converted DNA molecule comprising one or more loci of interest. In another embodiment, the unmethylated cytosine is converted into uracil via bisulfite treatment.

In still another embodiment, the method further comprises amplifying the cytosine-converted DNA molecule comprising one or more loci of interest, thereby generating an amplified double stranded DNA molecule comprising one or more loci of interest. In yet another embodiment, the amplification comprises polymerase chain reaction (PCR).

In one embodiment, the method further comprises operably linking a double stranded oligonucleotide comprising a first universal primer and a first sequencing primer to the first adaptor and a second double stranded oligonucleotide comprising a second universal primer and a second barcode to the second adaptor, wherein the nucleotide sequence of the first universal primer and the second universal primer is different.

In another embodiment, the amplified double stranded DNA molecule comprising one or more loci of interest, the first universal primer, and the first sequencing primer are operably linked in the followed order: 5′-the first universal primer-the first sequencing primer-the cytosine converted DNA-3′.

In still another embodiment, the first universal primer comprises a nucleotide sequence having about at least 85%, about 90%, about 91%, about 92%, about 93%, about 94%, about 95%, about 96%, about 97%, about 98%, about 99%, or about 100% nucleotide identity to the entire nucleotide sequence of SEQ ID NO: 10.

In one embodiment, the first sequencing primer is between 15 base pair to 30 base pair in length. In one embodiment, the first sequencing primer is 15, 16, 17, 18, 19, 20, 21, 22, 23, 24, 25, 26, 27, 28, 29, or 30 base pair in length. In another embodiment, the first sequencing primer comprises a nucleotide sequence having about at least 85%, about 90%, about 91%, about 92%, about 93%, about 94%, about 95%, about 96%, about 97%, about 98%, about 99%, or about 100% nucleotide identity to the entire nucleotide sequence of SEQ ID NO: 12.

In another embodiment, the amplified double stranded DNA molecule comprising one or more loci of interest, the second universal primer, and the second barcode are operably linked in the following order: 5′-the second universal primer-the second barcode-the cytosine converted DNA. In still another embodiment, the second universal primer comprises a nucleotide sequence having about at least 85%, about 90%, about 91%, about 92%, about 93%, about 94%, about 95%, about 96%, about 97%, about 98%, about 99%, or about 100% nucleotide identity to the entire nucleotide sequence of SEQ ID NO: 11. In yet another embodiment, the second barcode is between 6 nucleotides and 15 nucleotides in length. In one embodiment, the second barcode has a length of 8 nt.

In still another embodiment, the first double stranded oligonucleotide and the second double stranded oligonucleotide are operably linked to the cytosine-converted DNA by PCR.

In one aspect, the present invention provides a method for determining the methylation status of a loci of interest. The method includes preparing an amplified double stranded DNA molecule comprising one or more loci of interest according to the method of any one embodiment of any one of the above aspects and sequencing the double stranded DNA molecule, thereby determining the methylation status of the loci of interest.

In another aspect, the present invention provides a method for constructing a sequencing library for determining the methylation status of one or more loci of interest. The method includes fragmenting genomic DNA comprising one or more loci of interest to generate a plurality of double strand DNA molecules, wherein at least one of the plurality of double stranded DNA molecules comprises the one or more loci of interest; and preparing the plurality of double stranded DNA molecules comprising the one or more loci of interest according to any one embodiment of any one of the above aspects, thereby generating a sequencing library for determining the methylation status of one or more loci of interest. In one embodiment, the genomic DNA is human genomic DNA.

In some embodiments, the methods described herein comprise determining the methylation status of nucleic acids, e.g., genomic DNA, e.g., human genomic DNA. In some embodiments, the genomic DNA may be included in an amount less than about 10 pg. In some embodiments, the genomic DNA may be included in an amount greater than about 10 pg. In some embodiments, the genomic DNA may be included in an amount between about 0.25 to about 10 pg (e.g., about 0.25 pg, about 0.5 pg, about 0.75 pg, about 1 pg, about 1.25 pg, about 1.5 pg, about 1.75 pg, about 2 pg, about 2.25 pg, about 2.5 pg, about 2.75 pg, about 3 pg, about 3.25 pg, about 3.5 pg, about 3.75 pg, about 4 pg, about 4.25 pg, about 4.5 pg, about 4.75 pg, about 5 pg, about 5.25 pg, about 5.5 pg, about 5.75 pg, about 6 pg, about 6.25 pg, about 6.5 pg, about 6.75 pg, about 7 pg, about 7.25 pg, about 7.5 pg, about 7.75 pg, about 8 pg, about 8.25 pg, about 8.5 pg, about 8.75 pg, about 9 pg, about 9.25 pg, about 9.5 pg, about 9.75 pg, or about 10 pg).

In some embodiments, the genomic DNA may be included in an amount less than about 1 ng. In some embodiments, the genomic DNA may be included in an amount greater than about 1 ng. In some embodiments, the genomic DNA may be included in an amount between about 1 and about 50 ng (e.g., about 1 ng, about 2 ng, about 3 ng, about 4 ng, about 5 ng, about 6 ng, about 7 ng, about 8 ng, about 9 ng, about 10 ng, about 11 ng, about 12 ng, about 13 ng, about 14 ng, about 15 ng, about 16 ng, about 17 ng, about 18 ng, about 19 ng, about 20 ng, about 21 ng, about 22 ng, about 23 ng, about 24 ng, about 25 ng, about 26 ng, about 27 ng, about 28 ng, about 29 ng, about 30 ng, about 31 ng, about 32 ng, about 33 ng, about 34 ng, about 35 ng, about 36 ng, about 37 ng, about 38 ng, about 39 ng, about 40 ng, about 41 ng, about 42 ng, about 43 ng, about 44 ng, about 45 ng, about 46 ng, about 47 ng, about 48 ng, about 49 ng, or about 50 ng). In some embodiments, the genomic DNA may be included in an amount greater than about 50 ng.

In still another aspect, the present invention provides a method of determining the methylation status of one or more loci of interest. The method includes preparing a sequencing library according to the method of any one embodiment of any above aspects; and sequencing the one or more loci of interest; thereby determining the methylation status of one or more loci of interest.

In yet another aspect, the present invention provides a method of determining the methylation status of one or more loci of interest present in a plurality of subject samples. The method includes constructing a sequencing library from each subject according to the method of any one embodiment of the any one of the above aspects, wherein each library comprises a plurality of the double stranded DNA molecules comprising one or more loci of interest and wherein each of the first barcodes in each of the plurality of libraries is a unique first bar code; pooling the plurality of libraries; and sequencing the plurality of double stranded DNA molecules comprising one or more loci of interest; thereby determining the methylation status of one or more loci of interest present in the plurality of subject samples. In one embodiment, each of the second barcodes in each of the plurality of libraries is a unique second bar code

In one embodiment, the method further comprises comparing the methylation status of one or more loci of interest to a reference methylation status. In another embodiment, the comparison comprises comparison of the number of nucleotides comprising a methylated cytosine, the location of the methylated cytosine, or both.

In still another aspect, the present invention provides a method of predicting age. The method includes preparing a sequencing library according to the method of any one embodiment of any above aspects; sequencing the one or more loci of interest; applying an algorithm to create a linear model to predict methylation from age using a previously described bulk sequenced dataset; and taking a maximum likelihood approach to predict age from the sequencing data, thereby determining age. In certain embodiments, the sequencing is by shallow sequencing, which may comprise coverage of about 1 to about 2 reads per CpG. In certain embodiments, shallow sequencing may comprise <1 million reads per pool, for example, at each target locus. In certain embodiments, the algorithm is a scAge algorithm or a modified version thereof.

In yet another aspect, the present invention provides a method of determining the age of a plurality of subject samples. The method includes constructing a sequencing library from each subject according to the method of any one embodiment of the any one of the above aspects, wherein each library comprises a plurality of the double stranded DNA molecules comprising one or more loci of interest and wherein each of the first barcodes in each of the plurality of libraries is a unique first bar code; pooling the plurality of libraries; sequencing the plurality of double stranded DNA molecules comprising one or more loci of interest; applying an algorithm to create a linear model to predict methylation from age using a previously described bulk sequenced dataset; and taking a maximum likelihood approach to predict age from the sequencing data, thereby determining the age of the plurality of subject samples. In certain embodiments, the sequencing is by shallow sequencing, which may comprises coverage of about 1 to about 2 reads per CpG. In certain embodiments, shallow sequencing may comprise <1 million reads per pool, for example, at each target locus. In certain embodiments, the algorithm is a scAge algorithm or a modified version thereof.

In one embodiment, the method further comprises comparing the methylation status of one or more loci of interest to a reference methylation status. In another embodiment, the comparison comprises comparison of the number of nucleotides comprising a methylated cytosine, the location of the methylated cytosine, or both.

In one aspect, the present invention provides a kit for preparing a double stranded DNA molecule comprising one or more loci of interest for determining the methylation status of the one or more loci of interest therein. The kit includes a first partially double stranded oligonucleotide comprising a first adaptor single stranded oligonucleotide and a first barcode single stranded oligonucleotide; and a second partially double stranded oligonucleotide comprising second adaptor; wherein the first adaptor and the nucleotide sequence of the first barcode do not comprise a cytosine, wherein the second adaptor does not comprise a cytosine or the cytosine thereon is methylated; and wherein the first adaptor and the first barcode are operably linked, from 5′-terminus to 3′-terminus in the following order, the first adaptor-the first barcode.

In one embodiment, the first partially double stranded oligonucleotide further comprises a first enzyme recognition sequence, wherein the first enzyme recognition sequence is operably linked to the 3′-terminus of the first barcode; and wherein the second partially double stranded oligonucleotide further comprises a second enzyme recognition sequence, wherein the second enzyme recognition sequence is operably linked to the 3′-terminus of the second adaptor.

In another embodiment, the first enzyme recognition sequence and the second enzyme recognition sequence are specific site that an enzyme recognizes, and wherein the enzyme catalyzes the insertion of the first partially double stranded DNA and the second partially double stranded DNA to the 5′-terminus and 3′-terminus of a double stranded DNA molecule, respectively. In still another embodiment, the kit further comprises the enzyme.

In yet another embodiment, the first enzyme recognition sequence is a first end sequence for a transposon, the second enzyme recognition sequence is a second end sequence for the transposon, and the enzyme is a transposase. In one embodiment, the transposon is transposon 5 (Tn5) and the transposase is a hyperactive transposase Tn5. In another embodiment, the end sequence comprises a hyperactive mosaic end (ME) nucleotide sequence.

In one embodiment, the first partially double stranded oligonucleotide further comprises a first barcode.

### BRIEF DESCRIPTION OF THE DRAWINGS

FIGS. 1A-1D are schematics depicting exemplary compositions and methods for determining the methylation status of one or more loci of interest present in a deoxyribonucleic acid (DNA) molecule.

FIG. 1A depicts transposome assembly which includes combining a barcoded, cytosine-depleted adaptor A (the first adaptor) and a methylated adaptor B (the second adaptor).

FIG. 1B depicts that sample tagmentation of genomic DNA in multi-well format enables rapid and cost-effective barcoding and fragmentation of dozens to hundreds of samples simultaneously. After tagmentation, the samples are pooled and processed in one tube.

FIG. 1C depicts that methylated end-repair protects the reverse strand of both adaptors from sequence conversions, followed by target enrichment of loci of interest for DNA methylation sequencing, bisulfite conversion of DNA, and PCR in preparation for sequencing.

FIG. 1D depicts the production of RNA-baits for custom sequence enrichment. These baits are inexpensive and able to be continually regenerated from DNA template pools.

FIG. 2 is a schematic depicting an exemplary barcoded tagmentation and custom sequencing strategy for immediate pooling and bisulfite sequencing library preparation.

FIG. 3A is a schematic of a proof-of-concept sequencing experiment.

FIGS. 3B and 3C depict demultiplexing of all 16 samples after sequencing (FIG. 3B) and percent of reads that mapped to the mouse and lambda genomes (FIG. 3C).

FIG. 3D shows sequencing coverage of bisulfite sequencing reads across the lambda phage genome.

FIG. 3E shows the percentage of CpG methylation observed versus the percentage of CpG methylation expected from mouse bisulfite sequencing methylation standard DNA.

FIGS. 4A-4C are graphs depicting the results of targeted enrichment after tagmentation and pooling with the library preparation methods of the invention.

FIGS. 4A and 4B depict that each of the 12 samples demultiplexed (FIG. 4A) and a substantial percent of the reads mapped to the target loci (FIG. 4B). The top and bottom panels of FIG. 4A present the same data in two different formats. Similarly, the top and bottom panels of FIG. 4B present the same data in two different formats.

FIG. 4C depicts that the coverage across each target locus was roughly Poisson and sufficient for calculating the mouse blood DNA methylation clock.

FIG. 5 depicts the qPCR for determining the number of cycles for amplifying the DNA comprising one or more loci of interest.

FIGS. 6A-6E depicts exemplary Tagmentation-based Indexing for Methylation Sequencing (TIME-Seq), which enables low-cost and targeted bisulfite sequencing for biomarker measurement and discovery. TIME-Seq is a novel method that leverages (FIG. 6A) bisulfite-resistant transposomes to (FIG. 6B) barcode and fragment individual DNA samples, which are pooled for (FIG. 6C) methylated end-repair, target CpG enrichment, bisulfite conversion, and library amplification. FIG. 6D depicts that the samples are sequenced, demultiplexed, and DNA methylation values are calculated from mapped reads. FIG. 6E depicts DNA methylation-based biomarkers are analyzed or built using the methylation matrices from samples. Epigenetic clock analysis is shown as an example.

FIGS. 7A-7D depict graphical illustrations of TIME-Seq library preparation and sequencing. FIGS. 7A and 7C show exemplary library preparations with the sequence of various exemplary oligonucleotides indicated. FIG. 7B shows exemplary sequencing of a TIME-Seq library using a 150 cycle Illumina sequencing kit (e.g., a MiSeq version 3 kit).

FIG. 7D shows exemplary sequencing of a TIME-Seq library using a custom sequencing primer. Not shown is DNA clean up steps.

FIG. 7E depicts the sequences of various oligonucleotides that may be used in accordance with the methods described herein, for example, as shown in FIGS. 7A-7D.

FIGS. 8A-8D depicts the validation of demultiplexing, replicate correlation, and methylation accuracy of TIME-Seq libraries. FIG. 8A depicts reads demultiplexed by the internal Tn5-barcode from a single pool of 64 samples. The triangle is unidentified reads. FIG. 8B depicts average percent CpG methylation from mice of various ages and sexes in addition to mCpG standards. FIG. 8C depicts CpG methylation correlation between replicates in two separate TIME-Seq libraries. Coverage of at least 100. FIG. 8D depicts correlation between replicates using different coverage cutoffs.

FIGS. 9A-9E depicts efficient hybridization enrichment is compatible with TIME-Seq libraries. FIG. 9A depicts the percent of reads mapping within 1 kilobase (kb) of the target loci from TIME-Seq libraries. Targets were ≈1600 and ≈950 windows of 250 base pairs within the mouse and human genomes that are described as epigenetic “clock” CpGs.

FIG. 9B depicts CpG coverage from shallow sequencing (<1 million reads per pool) at each target locus across chromosome 1 in the human genome. Each point at a locus represents a sample in the TIME-Seq pool. FIG. 9C depicts the density of coverage for the demultiplexed samples from all target CpGs. FIG. 9D depicts the percent of bisulfite converted reads mapping to the ribosomal DNA (rDNA) repeat from a TIME-Seq library enriched with RNA-baits targeting rDNA. FIG. 9E depicts an IGV genome browser picture of read coverage pileup showing target loci (triangles below the screen shot) and each sample from one TIME-Seq pool enriched for rDNA.

FIGS. 10A-10C depicts the comparison of long and short adapter strategies shows short TIME-Seq adapters yield higher enrichment. FIG. 10A depicts TIME-Seq short (38-nt) and long (60-nt) barcoded adapter design. FIG. 10B depicts the comparison of TIME-Seq reads mapped within 2 kb of the targeted CpGs, enriched using baits complementary to a previously described mouse blood methylation clock. FIG. 10C depicts the percent of reads mapped to repetitive ribosomal DNA (rDNA) using short or long adapter design.

FIGS. 11A-11C depicts the characterization of TIME-Seq hybridization enrichment. FIGS. 11A-11B depicts the comparison of hybridization time and temperature conditions. FIG. 11C depicts the comparison of various library preparation conditions using mouse blood hybridization baits (24-hour hybridization).

FIGS. 12A-12E depicts the TIME-Seq ribosomal DNA methylation clock accurately predicts age. FIGS. 12A-12B depicts the age and sample count for the training and testing split of samples to build and test the rDNA methylation TIME-Seq clock. FIG. 12C depicts the age predications for training (closed circle) and testing (open circle). Sequencing cost for all 181 samples is approximately $5 per sample. FIG. 12D depicts an independent TIME-Seq library with longitudinal data to validate the TIME-Seq rDNA clock. FIG. 12E depicts the application of a TIME-Seq rDNA clock from publicly available RRBS data.

FIGS. 13A-13E depicts the highly accurate age prediction from shallow-sequencing using TIME-Seq. FIG. 13A depicts the experimental design for shallow sequencing data production. TIME-Seq libraries were enriched for previously described clock CpGs. Sequencing cost was less than $2 per sample. FIG. 13B depicts the demultiplexed reads from the 119 mouse blood samples showing only 50-30K reads per sample. FIG. 13C is an illustration of the general steps for shallow sequencing prediction using the scAge algorithm. FIG. 13D depicts the percent of reads that intersect CpGs included in the scAge model from TIME-Seq libraries and an example single cell dataset. This data demonstrate the advantage of using a TIME-Seq targeted approach. FIG. 13E depicts the predicted age (DNAme age) versus the chronological age from shallow sequencing data by scAge in both male and female mice. Highly accurate age estimations can be made using the methods described herein.

### DETAILED DESCRIPTION OF THE INVENTION

The present invention is based upon, at least partly, the discovery that the methylation status of one or more loci of interest on a deoxyribonucleic acid (DNA) molecule can be determined with high level of fidelity using a method that includes the use of cytosine free oligonucleotides as adaptors. Accordingly, the present invention provides compositions, kits, and methods for determining the methylation status of one or more loci of interest present in a DNA molecule. The methods of the present invention provide several advantages, including, but not limited to, low cost, and compatibility with sample multiplexing.

### I. Definitions

In order that the present invention may be more readily understood, certain terms are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately” means within 5%, or more preferably within 1%, of a given value or range.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

The term “adaptor” refers to a single stranded nucleic acid molecule that can be joined, i.e., operably linked, either using a ligase or a transposase-mediated reaction, to at least one strand of a double-stranded DNA molecule.

The term “amplifying” as used herein refers to the process of synthesizing copies of nucleic acid molecules that are complementary to one or both strands of a template nucleic acid.

Amplifying a nucleic acid molecule may include denaturing the template nucleic acid, annealing primers to the template nucleic acid at a temperature that is below the melting temperatures of the primers, providing free nucleotides, and enzymatically elongating from the primers to generate an amplification product. The denaturing, annealing and elongating steps each can be performed one or more times. In certain cases, the denaturing, annealing and elongating steps are performed multiple times such that the amount of amplification product is increased, often times exponentially, although exponential amplification is not required by the present methods. Amplification typically requires the presence of deoxyribonucleoside triphosphates, a DNA polymerase enzyme and an appropriate buffer and/or co-factors for optimal activity of the polymerase enzyme. The term “amplification product” refers to the nucleic acid molecules which are produced from the amplifying process as defined herein.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assessing may be relative or absolute.

The terms “barcode sequence” or “molecular barcode” or “barcode,” as used herein, refer to a single stranded nucleic acid molecule comprising a unique sequence of nucleotides used to a) identify and/or track the source of a polynucleotide present in a plurality of polynucleotides and/or b) count how many times an initial molecule is sequenced (e.g., in cases where substantially every molecule in a sample is tagged with a different sequence, and then the sample is amplified). A barcode sequence may be at the 5 ‘-end, the 3’-end or in the middle of a single stranded oligonucleotide, or both the 5′ end and the 3′ end. Barcode sequences may vary widely in size and composition; the following references provide guidance for selecting sets of barcode sequences appropriate for particular embodiments: Brenner, U.S. Pat. No. 5,635,400; Brenner et al, Proc. Natl. Acad. Sci., 97: 1665-1670 (2000); Shoemaker et al, Nature Genetics, 14: 450-456 (1996); Morris et al, European patent publication 0799897A1; Wallace, U.S. Pat. No. 5,981,179; and the like. In particular embodiments, a barcode may comprise a nucleotide sequence having a length in the range of from 4 to 36 nucleotides, or from 6 to 30 nucleotides, or from 8 to 24 nucleotides, or from 10 to 18 nucleotides. In some embodiments, a barcode sequence has length of 5, or 6, or 8 nucleotides.

“Blunt” or “blunt end” means that there are no unpaired nucleotides at that end of a double stranded nucleic acid molecule. A “blunt ended” double stranded DNA molecule may be double stranded over its entire length, i.e., no nucleotide overhang at either end of the molecule, or blunt ended on only one end of the molecule.

If two nucleic acids are “complementary”, they hybridize with one another under high or moderate stringency conditions. The terms “perfect complementarity” or “fully complementary” are used to describe a duplex in which each base of one of the nucleic acid molecules in the duplex base pairs with a complementary nucleotide in the second nucleic acid molecule in the duplex. In many cases, two sequences that are complementary have at least 10, e.g., at least 12 or 15 nucleotides of complementarity. The term “strand” as used herein refers to a nucleic acid molecule comprised of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure.

As used herein, the term “region of complementarity” refers to the region on one nucleic acid molecule that is substantially complementary to a sequence on another nucleic acid molecule. Where the region of complementarity is not fully complementary to the target sequence, the mismatches can be in the internal or terminal regions of the molecule. In some embodiments, a region of complementarity includes one or more nucleotide mismatches.

A double stranded nucleic acid molecule, as described herein, can contain about 1%, about 2%, about 3%, about 5%, about 6%, about 7%, about 8%, about 9%, about 10%, about 11%, about 12%, about 13%, about 14%, or about 15% mismatch. To determine the percentage of mismatch, the sequences are aligned so that the highest order match is obtained. Methods for determining mismatches are known and can be determined by commercially available computer programs that can calculate the percentage of identity between two or more sequences. A typical example of such a computer program is CLUSTAL. As an illustration, by a polynucleotide having a nucleotide sequence having at least, for example, 10% mismatch to a reference complementary polynucleotide is intended that the nucleotide sequence of the polynucleotide is complementary to the reference sequence except that the polynucleotide sequence may include on average of up to 10 mismatches per each 100 nucleotides of the reference nucleotide sequence. These mismatches may occur at the 5′ or 3′ terminal positions of the reference nucleotide sequence or anywhere between those terminal positions, interspersed either individually among nucleotides in the reference sequence or in one or more contiguous groups within the reference sequence.

The term “ligating”, as used herein, refers to the enzymatically catalyzed operably linking of the terminal nucleotide at the 5′ end of a first DNA molecule to the terminal nucleotide at the 3′ end of a second DNA molecule.

The term “hybridization” or “hybridizes” refers to a process in which a single stranded nucleic acid molecule anneals to and forms a stable duplex, either a homoduplex or a heteroduplex, under normal hybridization conditions with a second complementary single stranded nucleic acid molecule, and does not form a stable duplex with unrelated nucleic acid molecules under the same normal hybridization conditions. The formation of a duplex is accomplished by annealing two complementary single stranded nucleic acid molecules in a hybridization reaction. The hybridization reaction can be made to be highly specific by adjustment of the hybridization conditions (often referred to as hybridization stringency) under which the hybridization reaction takes place, such that hybridization between two nucleic acid molecules will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

As used herein “multiplex amplification” refers to selective and non-random amplification of two or more target sequences within a sample using at least one target-specific primer. In some embodiments, multiplex amplification is performed such that some or all of the target sequences are amplified within a single reaction vessel. The “plexy” or “plex” of a given multiplex amplification refers generally to the number of different target-specific sequences that are amplified during that single multiplex amplification. In some embodiments, the plexy can be about 12-plex, 24-plex, 48-plex, 96-plex, 192-plex, 384-plex, 768-plex, 1536-plex, 3072-plex, 6144-plex or higher. It is also possible to detect the amplified target sequences by several different methodologies (e.g., gel electrophoresis followed by densitometry, quantitation with a bioanalyzer or quantitative PCR, hybridization with a labeled probe; incorporation of biotinylated primers followed by avidin-enzyme conjugate detection; incorporation of ³²P-labeled deoxynucleotide triphosphates into the amplified target sequence).

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes. The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., peptide nucleic acid or PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylenecarbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid may contain a G′ residue and a C residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

The term “oligonucleotide” as used herein denotes a multimer of nucleotides of from about 2 to 200 nucleotides, or up to 500 nucleotides in length.

Oligonucleotides may be synthetic or may be made enzymatically, and, in some embodiments, are 10 to 150 nucleotides in length. Oligonucleotides may contain ribonucleotide monomers (i.e., may be oligoribonucleotides) or deoxyribonucleotide monomers, or both ribonucleotide monomers and deoxyribonucleotide monomers. An oligonucleotide may be 10 to 20, 11 to 30, 31 to 40, 41 to 50, 51 to 60, 61 to 70, 71 to 80, 80 to 100, 100 to 150 or 150 to 200 nucleotides in length, for example. An oligonucleotide may be double stranded, single stranded, or partially double stranded.

The terms “operably linked”, “in operable combination”, and “in operable order” refer to the linkage of nucleic acid sequences in such a manner that they are suitably positioned and oriented for, e.g., transcription to be initiated.

A “plurality” contains at least 2 members. In certain cases, a plurality may have at least 2, at least 5, at least 10, at least 100, at least 100, at least 10,000, at least 100,000, at least 10⁶, at least 10⁷, at least 10⁸ or at least 10⁹ or more members.

“Primer” refers to a single stranded oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with their use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18 to 40, 20 to 35, 21 to 30 nucleotides long, and any length between the stated ranges. Typical primers can be in the range of between 10 to 50 nucleotides long, such as 15 to 45, 18 to 40, 20 to 30, 21 to 25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length. Thus, a “primer” is complementary to a template, and complexes with the template to give a primer/template complex for initiation of synthesis by a polymerase, which is extended by the addition of covalently bonded bases linked at its 3′ end complementary to the template in the process of DNA synthesis.

The term “sample” as used herein relates to a material or mixture of materials, typically, although not necessarily, in liquid form, containing one or more analytes of interest. The nucleic acid samples used herein may be complex in that they contain multiple different molecules that contain sequences. Genomic DNA and cDNA made from mRNA from a mammal (e.g., mouse or human) are types of complex samples. Complex samples may have more then 1, 10, 10², 10³, 10⁴, 10⁵, 10⁶, or 10⁷ different nucleic acid molecules. A DNA target may originate from any source such as genomic DNA, cDNA (from RNA) or artificial DNA constructs. Any sample containing nucleic acid, e.g., genomic DNA made from tissue culture cells, a sample of tissue, or an FFPE samples, may be employed herein. In some embodiments, a sample may comprise nucleic acids which are not contained within an isolated nuclei.

In some embodiments, a sample may comprise nucleic acids, e.g., genomic DNA, e.g., human genomic DNA. In some embodiments, a sample may comprise nucleic acids, e.g., genomic DNA, e.g., human genomic DNA in an amount less than about 10 pg. In some embodiments, a sample may comprise nucleic acids, e.g., genomic DNA, e.g., human genomic DNA in an amount greater than about 10 pg. In some embodiments, a sample may comprise nucleic acids, e.g., genomic DNA, e.g., human genomic DNA in an amount between about 0.25 to about 10 pg (e.g., about 0.25 pg, about 0.5 pg, about 0.75 pg, about 1 pg, about 1.25 pg, about 1.5 pg, about 1.75 pg, about 2 pg, about 2.25 pg, about 2.5 pg, about 2.75 pg, about 3 pg, about 3.25 pg, about 3.5 pg, about 3.75 pg, about 4 pg, about 4.25 pg, about 4.5 pg, about 4.75 pg, about 5 pg, about 5.25 pg, about 5.5 pg, about 5.75 pg, about 6 pg, about 6.25 pg, about 6.5 pg, about 6.75 pg, about 7 pg, about 7.25 pg, about 7.5 pg, about 7.75 pg, about 8 pg, about 8.25 pg, about 8.5 pg, about 8.75 pg, about 9 pg, about 9.25 pg, about 9.5 pg, about 9.75 pg, or about 10 pg).

In some embodiments, a sample may comprise nucleic acids, e.g., genomic DNA, e.g., human genomic DNA in an amount less than about 1 ng. In some embodiments, a sample may comprise nucleic acids, e.g., genomic DNA, e.g., human genomic DNA in an amount greater than about 1 ng. In some embodiments, a sample may comprise nucleic acids, e.g., genomic DNA, e.g., human genomic DNA in an amount between about 1 and 50 ng (e.g., about 1 ng, about 2 ng, about 3 ng, about 4 ng, about 5 ng, about 6 ng, about 7 ng, about 8 ng, about 9 ng, about 10 ng, about 11 ng, about 12 ng, about 13 ng, about 14 ng, about 15 ng, about 16 ng, about 17 ng, about 18 ng, about 19 ng, about 20 ng, about 21 ng, about 22 ng, about 23 ng, about 24 ng, about 25 ng, about 26 ng, about 27 ng, about 28 ng, about 29 ng, about 30 ng, about 31 ng, about 32 ng, about 33 ng, about 34 ng, about 35 ng, about 36 ng, about 37 ng, about 38 ng, about 39 ng, about 40 ng, about 41 ng, about 42 ng, about 43 ng, about 44 ng, about 45 ng, about 46 ng, about 47 ng, about 48 ng, about 49 ng, or about 50 ng). In some embodiments, a sample may comprise nucleic acids, e.g., genomic DNA, e.g., human genomic DNA in an amount greater than about 50 ng.

In some embodiments, a sample may comprise isolated nuclei.

In some embodiments, a sample may be substantially free of isolated nuclei.

In some embodiments, a sample may not comprise isolated nuclei.

The term “sequencing”, as used herein, refers to a method by which the identity of at least 10 consecutive nucleotides (e.g., the identity of at least 20, at least 50, at least 100 or at least 200 or more consecutive nucleotides) of a polynucleotide are obtained. In certain embodiments, the term “sequencing” may be used to refer to next-generation sequencing.

The term “next-generation sequencing” refers to the parallelized sequencing-by-synthesis or sequencing-by-ligation platforms currently employed by Illumina, Life Technologies, and Roche etc. Next-generation sequencing methods may also include nanopore sequencing methods or electronic-detection based methods such as Ion Torrent technology commercialized by Life Technologies.

As used herein, the term “target,” when used in reference to a nucleic acid, is intended as a semantic identifier for the nucleic acid in the context of a method or composition set forth herein and does not necessarily limit the structure or function of the nucleic acid beyond what is otherwise explicitly indicated. A target nucleic acid may be essentially any nucleic acid of known or unknown sequence. It may be, for example, a fragment of genomic DNA or cDNA. Sequencing may result in determination of the sequence of the whole, or a part of the target molecule. The targets can be derived from a primary nucleic acid sample, such as a nucleus. In one embodiment, the targets can be processed into templates suitable for amplification by the placement of universal sequences at the ends of each target fragment. The targets can also be obtained from a primary RNA sample by reverse transcription into cDNA.

As used herein, a “transposome complex” refers to an integration enzyme and a nucleic acid molecule which includes an integration recognition site. A “transposome complex” is a functional complex formed by a transposase and a transposase recognition site that is capable of catalyzing a transposition reaction (see, for instance, Gunderson et al., WO 2016/130704). Examples of integration enzymes include, but are not limited to, such as an integrase or a transposase. Examples of integration recognition sites include, but are not limited to, a transposase recognition site, and a transposon end sequence.

The term “transposon end sequence” refers to a double-stranded or partially double-stranded sequence to which a transposase (e.g., Tn5 transposase or variant thereof) binds, where the transposase catalyzes simultaneous fragmentation of a double-stranded DNA sample and tagging of the fragments with sequences that are adjacent to the transposon end sequence, e.g., the adaptor and/or the barcode (i.e., by “tagmentation”). Methods for tagmenting and transposon end sequences are well known in the art (see, e.g., Picelli et al, Genome Res. 2014 24: 2033-40; Adey et al, Genome Biol. 2010 11:R119 and Caruccio et al, Methods Mol. Biol. 2011 733: 241-55, US20100120098 and US20130203605). Kits for performing tagmentation are commercially sold under the tradename NEXTERA™ by Illumina (San Diego, Calif.). The double-stranded form of AGA TGT GTA TAA GAG ACA G (SEQ ID NO: 1) is an example of a Tn5 transposon end sequence, although many others are known and are typically 18-20 bp, e.g., 19 bp in length.

As used herein, the term “universal,” when used to describe a nucleotide sequence, refers to a region of sequence that is common to two or more nucleic acid molecules where the molecules also have regions of sequence that differ from each other. A universal sequence that is present in different members of a collection of molecules can allow capture of multiple different nucleic acids using a population of universal capture nucleic acids, e.g., capture oligonucleotides, that are complementary to a portion of the universal sequence, e.g., a universal capture sequence. Non-limiting examples of universal capture sequences include sequences that are identical to or complementary to P5 and P7 primers. Similarly, a universal sequence present in different members of a collection of molecules can allow the replication or amplification of multiple different nucleic acids using a population of universal primers that are complementary to a portion of the universal sequence, e.g., a universal anchor sequence. A capture oligonucleotide or a universal primer therefore includes a sequence that can hybridize specifically to a universal sequence.

### II. Methods of the Invention

DNA methylation is a biological process by which methyl groups are added to a DNA molecule. Methylation can change the activity of a DNA segment without changing the nucleotide sequence of the segment. When located in a gene promoter, DNA methylation typically acts to repress gene transcription. In mammals, DNA methylation is essential for normal development and is associated with a number of key processes including genomic imprinting, X-chromosome inactivation, repression of transposable elements, aging, and carcinogenesis.

While two of DNA's four bases, cytosine and adenine, can be methylated, cytosine methylation is widespread in both eukaryotes and prokaryotes and has been extensively studied. In mammals, DNA methylation is almost exclusively found in CpG dinucleotides. Changes in DNA methylation status have been implicated in, inter alia, embryonic development, cancer, atherosclerosis, aging, immune system development, and central nervous system development. DNA methylation is attracting increasing attention as a potential biomarker. Methods to determine methylation status can be used for detection and diagnosis of disease, prediction of response to therapeutic interventions and prognosis of outcome.

Numerous methods have ben developed to determined the DNA methylation status of a genome, including, but are not limited to, mass spectrometry, methylation-specific PCR, sequencing based-assay such as bisulfite sequencing, the Hpall tiny fragment Enrichment by Ligation-mediated PCR (HELP) assay, GLAD-PCR assay, ChIP-on-chip assay, restriction landmark genomic scanning, methylated DNA immuneprecipitation, methyl sensitive southern blotting, high resolution Melt analysis, and methylation sensitive single nucleotide primer extension assay.

Currently, two assays are the dominant methods for assaying DNA methylation-based biomarkers such as DNA methylation clocks. For most human studies, Illumina microarray-based methylation chips (e.g., the Infinium MethylationEPIC Chip that measures DNA methylation at 850,000 CpGs) are used. These arrays are not compatible with sample multiplexing and cost upward of $280 per sample. Without being bound by theory, methylation microarrays are not compatible with sample multiplexing at least because they are not sequencing-based and do not produce any other secondary read-out for sample identification within a single microarray lane. In mouse studies, the most common method for methylation clock analysis is Reduced-Representation Bisulfite Sequencing (RRBS) (Meissner, A. et al. Reduced representation bisulfite sequencing for comparative high-resolution DNA methylation analysis. Nucleic acids research 33, 5868-5877, doi:10.1093/nar/gki901 (2005); Gu, H. et al. Preparation of reduced representation bisulfite sequencing libraries for genome-scale DNA methylation profiling. Nature protocols 6, 468-481, doi:10.1038/nprot.2010.190 (2011)). This method, while designed to increase sequencing coverage of CpG dense regions, nonetheless, has limited multiplexing capacity and suffers from substantial batch effects based on variable restriction enzyme digestion used to prepare libraries.

Bisulfite sequencing is also extensively used for determining DNA methylation status of DNA molecule. Bisulfite sequencing (also known as bisulphite sequencing) is the use of bisulfite treatment of DNA before routine sequencing to determine the pattern of methylation. Treatment of DNA with bisulfite converts cytosine residues to uracil, but leaves 5-methylcytosine residues unaffected. Therefore, DNA that has been treated with bisulfite retains only methylated cytosines. Thus, bisulfite treatment introduces specific changes in the DNA sequence that depend on the methylation status of individual cytosine residues, yielding single-nucleotide resolution information about the methylation status of a segment of DNA. Various analyses can be performed on the altered sequence to retrieve this information. The result of bisulfite sequencing is, therefore, merely differentiating between single nucleotide polymorphisms (cytosines and thymidine) resulting from bisulfite conversion.

As indicated above, currently available methods for determining DNA methylation status detect unmethylated cytosine that has been converted into uracil in a DNA molecule of interest. Thus, for detecting a converted cytosine using a next generation sequencing method, e.g., Illumina's Nextera, it is important that the adaptors and/or barcodes are resistant to the treatment, e.g., bisulfite treatment, that converts a cytosine present in a DNA molecule into uracil. Typically, the adaptors/barcodes used for methods in the art are synthesized using methylated cytosine and are, thus, resistant to the treatment that converts cytosine into uracil. The cost of such synthesized oligonucleotides that are fully cytosine-methylated is high. For example, a fully methylated, barcoded adaptor costs about $250 to $300.

The present invention is based upon, at least partly, the discovery that cytosine free oligonucleotides can be used as adaptors and/or barcodes to determine the methylation status of one or more loci of interest present in a population of a plurality of deoxyribonucleic acid (DNA) molecules with a high level of fidelity using next generation sequencing. Accordingly, the present invention provides methods of operably linking adaptors/barcodes that do not comprise a cytosine to DNA molecules for determining the DNA methylation status of one or more loci of interest present in a population of a plurality of DNA molecules using a next generation sequencing method. The adaptors and/or barcodes are typically single stranded deoxyribonucleic oligonucleotides.

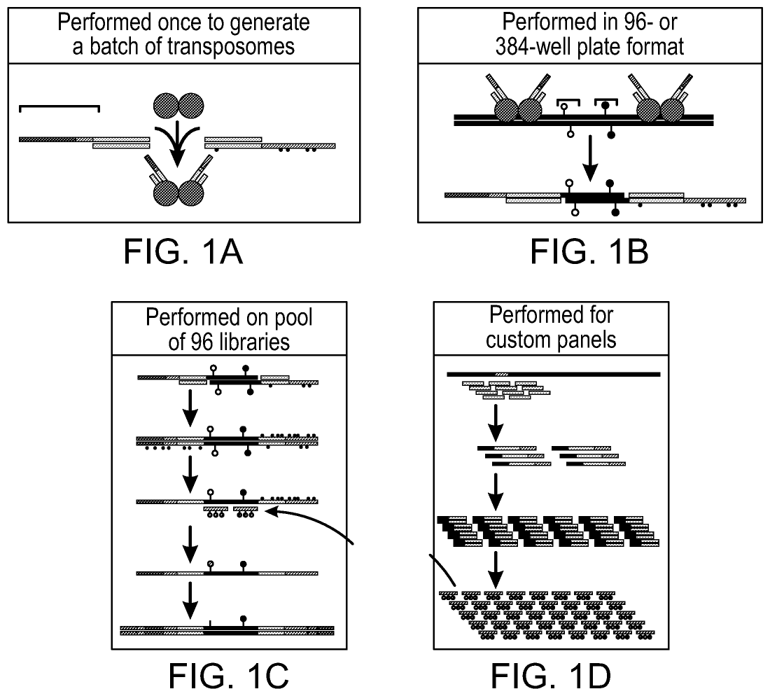

The methods of the present invention include, inter alia, using adaptors and/or barcodes that do not comprise a cytosine. As described herein and shown in FIG. 1, the present invention includes an adaptor/barcode that does not comprise a cytosine (i.e., cytosine-depleted adaptor/barcode). The adaptor may be of any suitable length. In some embodiments, the adaptor has a length of 14-nt followed 3′ by a barcode. The barcode may be of any suitable length. In some embodiments, the barcode has a length of 5 nt. The adaptor and the barcode that do not comprise a cytosine are operably linked in the order: 5′-adaptor-barcode-3′. In some embodiments, the present invention further features a sequencing primer that is added to the 5′-terminus of the adaptor that does not comprises a cytosine. As shown in FIG. 1, the primer for amplification from the cytosine-depleted adaptor A adds a sequence upstream that allows for a first sequencing primer to be added for sequencing of the barcode.

The present invention further features the combination of two adaptors that are resistant to a treatment that converts a cytosine to a uracil. In some embodiments, both adaptors do not comprise cytosine. In some embodiments, one of the two adaptors does not comprise cytosine and another one comprises a methylated cytosine. Such a combination of two adaptors are suitable for nucleotide sequencing using any suitable sequencing method known in the art used to determine the methylation status of one or more loci of interest on a DNA molecule, such as bisulfite sequencing.

The present invention also features the use of methylated end-repair to protect the reverse strand of the adaptors that are resistant to the conversion of cytosine to uracil, e.g., cytosine-depleted adaptor or cytosine-methylated adaptor.

The present invention further features a Tn5-based library preparation method that is compatible with bisulfite sequencing in combination with targeted sequence enrichment, such as RNA/DNA baits hybridization.

A. Methods for Assembling an Integrase and DNA Oligonucleotide Complex for Use in a Methods for Preparing a Double Stranded Deoxyribonucleic Acid (DNA) Molecule Comprising One or More Loci of Interest for Determining the Methylation Status of One or More Loci of Interest Therein

In one aspect, the present invention provides a method for assembling an enzyme-deoxyribonucleic acid (DNA) complex for use in preparing a double stranded DNA molecule comprising one or more loci of interest for determining the methylation status of the one or more loci of interest therein. The method includes contacting an enzyme with a first partially double stranded oligonucleotide comprising a first adaptor single stranded oligonucleotide and a first barcode single stranded oligonucleotide, wherein the first adaptor oligonucleotide and the first barcode oligonucleotide are operably linked in the order, from 5′ to 3′, the first adaptor-the first barcode, and a second partially double stranded oligonucleotide comprising a second adaptor single stranded oligonucleotide, wherein the enzyme is capable of operably linking the first and the second partially double stranded oligonucleotides to the double stranded DNA molecule comprising one or more loci of interest; wherein the first adaptor and the first barcode do not comprise a cytosine, wherein the second adaptor does not comprise a cytosine or the cytosine thereon is methylated; and wherein the nucleotide sequence of the first adaptor and the second adaptor are different, thereby preparing the enzyme-DNA complex.

In another aspect, the present invention provides a method of preparing a double stranded deoxyribonucleic acid (DNA) molecule comprising one or more loci of interest for determining the methylation status of one or more loci of interest therein. The method includes providing a double stranded DNA molecule comprising one or more loci of interest, contacting the double stranded DNA molecule comprising one or more loci of interest with the enzyme-DNA complex prepared according to any of the methods of the invention.

The first partially double stranded oligonucleotide may further comprise a first enzyme recognition sequence which is operably linked to the 3′-terminus of the first barcode; and the second partially double stranded oligonucleotide may further comprise a second enzyme recognition sequence, wherein the second enzyme recognition sequence is operably linked to the 3′-terminus of the second adaptor.

In some embodiments, the first enzyme recognition sequence is a first transposon end sequence of a transposon, and the second enzyme recognition sequence is a second transposon end sequence of the transposon. In certain embodiments, the first transposon end sequence and the second transposon sequence are double stranded DNA oligonucleotides.

In certain embodiments, the enzyme is an integrase. The term “end sequence,” as used herein, refers to a double-stranded or partially double-stranded sequence to which an integrase (e.g., a Tn5 transposase or variant thereof) binds, where the integrase catalyzes simultaneous fragmentation of a double-stranded DNA sample and tagging of the fragments with sequences that are adjacent to the end sequence, e.g., the adaptor and/or the barcode (i.e., by “tagmentation”).

Exemplary integrases include, but are not limited to, a transposase, and a retroviral integrase, such as integrases from HIV-1, HIV-2, SIV, PFV-1, or RSV. For retroviral integrases, the end sequences are integrase recognition sequences for such retroviral integrases.

In some embodiments, the integrase is a transposase.

The first or the second double stranded oligonucleotide complexed with a transposase is referred to as a “transposome” herein. Some embodiments of a transposome can include a hyperactive Tn5 transposase and a Tn5-type transposon end sequence (Goryshin and Reznikoff, J. Biol. Chem., 273:7367 (1998)), or MuA transposase and a Mu transposase recognition site comprising R1 and R2 end sequences (Mizuuchi, K., Cell, 35: 785, 1983; Savilahti, H, et al., EMBO J., 14: 4893, 1995). Tn5 Mosaic End (ME) sequences can also be used as optimized by a skilled artisan.

Additional examples of integrases and end sequences that can be used with certain embodiments of the compositions and methods provided herein include Staphylococcus aureus Tn552 (Colegio et al., J. Bacteriol., 183: 2384-8, 2001; Kirby C et al., Mol. Microbiol., 43: 173-86, 2002), Tyl (Devine & Boeke, Nucleic Acids Res., 22: 3765-72, 1994 and International Publication WO 95/23875), Transposon Tn7 (Craig, N L, Science. 271: 1512, 1996; Craig, N L, Review in: Curr Top Microbiol Immunol., 204:27-48, 1996), Tn/O and IS10 (Kleckner N, et al., Curr Top Microbiol Immunol., 204:49-82, 1996), Mariner transposase (Lampe D J, et al., EMBO J., 15: 5470-9, 1996), Tc1 (Plasterk R H, Curr. Topics Microbiol. Immunol., 204: 125-43, 1996), P Element (Gloor, G B, Methods Mol. Biol., 260: 97-114, 2004), Tn3 (Ichikawa & Ohtsubo, J Biol. Chem. 265:18829-32, 1990), bacterial insertion sequences (Ohtsubo & Sekine, Curr. Top. Microbiol. Immunol. 204: 1-26, 1996), retroviruses (Brown, et al., Proc Natl Acad Sci USA, 86:2525-9, 1989), and retrotransposon of yeast (Boeke & Corces, Annu Rev Microbiol. 43:403-34, 1989). More examples include IS5, Tn10, Tn903, IS911, and engineered versions of transposase family enzymes (Zhang et al., (2009) PLoS Genet. 5:e1000689. Epub 2009 Oct. 16; Wilson C. et al (2007) J. Microbiol. Methods 71:332-5).

Transposon end sequences useful with the methods and compositions described herein are provided in U.S. Patent Application Pub. No. 2012/0208705, U.S. Patent Application Pub. No. 2012/0208724 and Int. Patent Application Pub. No. WO 2012/061832, the entire contents of each of which are incorporated herein by reference.

In some embodiments, the transposon end sequence is a hyperactive Tn5 mosaic end sequence having the sequence AGA TGT GTA TAA GAG ACA G (SEQ ID NO: 1), or a variant thereof. In certain embodiments, the transposon end sequence is a double stranded oligonucleotide having a sense strand having the sequence of SEQ ID NO: 1, or a variant thereof, and an anti-sense strand having the sequence of SEQ ID NO: 2, or a variant thereof, a representative of which is shown in the below structure:

(SEQ ID NO: 1)
5′-AGATGTGTATAAGAGACAG-3′
(SEQ ID NO: 2)
3′-TCTACACATATTCTCTGTC-5′

In certain embodiments according to the present invention, adaptors/barcodes do not comprise a cytosine and, thus, are resistant to the treatment that converts unmethylated cytosine into uracil. The adaptors of the invention may have a length between 10 nucleotides (nt) to 30 nucleotides. In some embodiments, the adaptor may have a length between 14 nt to 20 nt. In some embodiments, the adaptor has a length of 15 nt. The adaptor has a sequence DDDDDDDDDDDDDDD (SEQ ID No. 3). The letter “D,” as used in sequence listing of the present invention, represents a nucleotide that is not cytosine. In some embodiments, the adaptor is a polynucleotide having a sequence of TGG GTG GAG GGT GG (SEQ ID NO: 4), or a variant thereof. The term “variant,” as used herein, refers to a polynucleotide that is derived by incorporation of one or more nucleotide insertions, substitutions, or deletions in a precursor polynucleotide (e.g., “parent” polynucleotide). In certain embodiments, a variant polynucleotide has at least about 85% nucleotide sequence identity, e.g., about 90%, about 91%, about 92%, about 93%, about 94%, about 95%, about 96%, about 97%, about 98%, about 99%, or about 100%, nucleotide sequence identity to the entire nucleotide sequence of a parent polynucleotide.

The term “sequence identity,” as used herein, refers to a comparison between pairs of nucleic acid or amino acid molecules, i.e., the relatedness between two amino acid sequences or between two nucleotide sequences. In general, the sequences are aligned so that the highest order match is obtained. Methods for determining sequence identity are known and can be determined by commercially available computer programs that can calculate the percentage of identity between two or more sequences. A typical example of such a computer program is CLUSTAL. In some embodiments, the barcode has a length between 4 nt and 10 nt. In some embodiments the barcode has a length of 5 nt or 6 nt. In certain embodiments, the barcode has a sequence DDDDD or DDDDDD.

In some embodiments, the adaptor and the barcode comprise methylated cytosine and, thus, resistant to the treatment that converts unmethylated cytosine into uracil. In some embodiment, the methylated adaptor is a polynucleotide having a sequence of GTmCTmCGTGGGmCTmCGG (SEQ ID NO: 5), or a variant thereof. In some embodiments, the adaptor and the barcode comprise methylated cytosine and, thus, resistant to the treatment that converts unmethylated cytosine into uracil. In some embodiment, the methylated adaptor is a polynucleotide having a sequence of TmCGTmCGGmCAGmCGTmC (SEQ ID NO: 16), or a variant thereof. As used in the present application, the symbol “mC” represents methylated cytosine.

In some embodiments, the adaptor and the barcode are operably linked according to the following order: 5′-adaptor-barcode-3′.

In certain embodiments, the partially double stranded oligonucleotide is formed by operably linking the adaptor and the barcode to an end sequence in the following order: 5′-adaptor-barcode-sense strand of end sequence-3′. In some embodiments where the adaptor comprises methylated cytosine, the partially double stranded oligonucleotide has the structure as follows: 5′-adaptor-sense strand end sequence-3′. In certain embodiments, the partially double stranded oligonucleotide comprises a top strand having the sequence of SEQ ID NO: 6, or a variant thereof, and a bottom strand having the sequence of SEQ ID NO: 2, or a variant thereof, a representative of which is shown in the below structure:

(SEQ ID NO: 6)
5′-TGGGTGGAGGGTGGDDDDDAGATGTGTATAAGAGACAG-3′
(SEQ ID NO: 2)
3′-TCTACACATATTCTCTGTC-5′

In certain embodiments, the partially double stranded oligonucleotide comprises a top strand having the sequence of SEQ ID NO: 21, or a variant thereof, and a bottom strand having the sequence of SEQ ID NO: 2, or a variant thereof, a representative of which is shown in the below structure:

(SEQ ID NO: 21)
5′-GTmCTmCGTGGGmCTmCGGAGATGTGTATAAGAGACAG-3′
(SEQ ID NO: 2)
3′-TCTACACATATTCTCTGTC-5′

In certain embodiments, the partially double stranded oligonucleotide comprises a top strand having the sequence of SEQ ID NO: 7, or a variant thereof, and a bottom strand having the sequence of SEQ ID NO: 2, or a variant thereof, a representative of which is shown in the below structure:

(SEQ ID NO: 7)
5′-GTmCTmCGTGGGmCTmCGGAGATGTGTATAAGAGAmCAG-3′
(SEQ ID NO: 2)
3′-TCTACACATATTCTCTGTC-5′

In certain embodiments, the partially double stranded oligonucleotide comprises a top strand having the sequence of SEQ ID NO: 17, or a variant thereof, and a bottom strand having the sequence of SEQ ID NO: 2, or a variant thereof, a representative of which is shown in the below structure:

(SEQ ID NO: 17)
5′-TmCGTmCGGmCAGmCGTmCAGATGTGTATAAGAGAmCAG-3′
(SEQ ID NO: 2)
3′-TCTACACATATTCTCTGTC-5′

The partially double stranded oligonucleotide and the integrase are assembled in a complex, e.g., transposome, for DNA tagmentation. The methods for assembling the partially double stranded oligonucleotide and integrase, e.g., transposase, are well known in the art (See, e.g., Adey and Shendure, Ultra-Low-Input, Tagmentation-based Whole-Genome Bisulfite Sequencing, Genome Research 22: 1139-42 (2012)).

### B. Methods for Preparing a Double Stranded Deoxyribonucleic Acid (DNA) Molecule Comprising One or More Loci of Interest for Determining the Methylation Status of One or More Loci of Interest Therein

In another aspect, the present invention provides a method of preparing a double stranded deoxyribonucleic acid (DNA) molecule comprising one or more loci of interest for determining the methylation status of the one or more loci of interest therein. The method includes providing a double stranded DNA molecule comprising one or more loci of interest, the DNA molecule comprising a first strand and a second strand; operably linking a first partially double stranded oligonucleotide comprising a first adaptor single stranded oligonucleotide and a first barcode single stranded oligonucleotide to the 5′-terminus of the first strand of the double stranded DNA molecule in the order, from 5′ to 3′, the first adaptor-the first barcode-the double strand DNA molecule; and operably linking a second partially double stranded oligonucleotide comprising a second adaptor single stranded oligonucleotide to the 5′-terminus of the second strand of the DNA molecule, wherein the first adaptor and the first barcode do not comprise a cytosine, wherein the second adaptor does not comprise a cytosine or the cytosine thereon is methylated; and wherein the nucleotide sequence of the first adaptor and the second adaptor are different, thereby preparing the double stranded DNA comprising one or more loci of interest for determining the methylation status of the one or more loci of interest therein.

The method may further comprise assembling a integrase-partially double stranded oligonucleotide complex, e.g., transposome, which includes contacting a integrase with the first partially double stranded oligonucleotide and the second partially double stranded oligonucleotide. In some embodiments, the integrase is a transposase and the transposase-partially double stranded oligonucleotide complex is a transposome.

Additionally, the method may further comprise contacting the transposome with the double stranded DNA molecule comprising one or more loci of interest, wherein the transposome fragments the double stranded DNA molecule comprising one or more loci of interest and operably links the first partially double stranded oligonucleotide and the second partially double stranded oligonucleotide to the double stranded DNA molecule comprising one or more loci of interest (i.e., tagmentation).

In some embodiment, the partially double stranded oligonucleotide comprises a Tn5 ME sequence and the integrase is transposase Tn5 or hyperactive transposase Tn5. The methods for DNA tagmentation is well known in the art (See, e.g., Adey and Shendure, supra).

In some embodiments, the DNA molecules comprising one or more loci of interest are fragmented with two partially double stranded oligonucleotide and integrase complexes, e.g., transposomes. In certain embodiments, the two partially double stranded oligonucleotides comprise different adaptor and barcode. In some embodiment, one of the two partially double stranded oligonucleotides comprises an adaptor and a barcode that comprise no cytosine and another partially double stranded oligonucleotide comprises an adaptor and a barcode that comprise methylated cytosine.

In some embodiments, the integrase-DNA oligonucleotide complex, e.g., transposome, contacts the DNA molecule comprising one or more loci of interest directly. In some embodiments, the integrase-DNA oligonucleotide complex, e.g., transposome, contacts the DNA molecule comprising one or more loci of interest indirectly. For example, an integrase may be engineered to bind to a DNA binding protein to mediate the tagmentation. An exemplary method is described in Kaya-Okur et al. (CUT & Tag for efficient epigenomic profiling of small samples and single cells, Nature Communications, 10: Article Number 1930 (2019)). In this method, a protein-A-Tn5 transposase fusion protein is used to facilitate targeted transposition by binding to an antibody that, in turn, binds to a DNA or chromatin binding protein. In certain embodiment, the DNA binding protein is Cas9 or dCas9. The Cas9 or dCas9-gRNA may target loci of interest on a DNA molecule. A protein-A-Tn5 fusion protein may target the loci of interest through an antibody that specifically recognize Cas9 or dCas9.

In certain embodiments, the first and the second partially double stranded oligonucleotides are operably linked to the DNA molecule comprising one or more loci of interest by ligation.

In addition, the method may further comprise repairing the ends of double stranded DNA molecule comprising one or more loci of interest operably linked to the first oligonucleotide and the second oligonucleotide using methylated cytosine, thereby generating an end repaired double stranded DNA comprising one or more loci of interest. Repairing the ends of the double stranded DNA molecule comprising one or more loci of interest operably linked to the first oligonucleotide and the second oligonucleotide may include the use of a Klenow, a T4 polymerase, or a mixture thereof.

In some embodiments, the method further comprises enriching the DNA molecule comprising one or more loci of interest (i.e., target enrichment) following end repairing, thereby generating enriched DNA comprising one or more loci of interest. Target-enrichment methods allow one to selectively capture genomic regions of interest from a DNA sample prior to sequencing. Several target-enrichment strategies have been developed since the original description of the direct genomic selection (DGS) method in 2005 (Basiardes, et al., Direct Genomic Selection, Nature Methods, 1(2): 63-69 (2005)). Target enrichment methods include array-based capture and in-solution based capture.

In array-based capture, microarrays contain single-stranded oligonucleotides with sequences from the genome to tile the region of interest fixed to the surface. The DNA molecules comprising one or more loci of interest are hybridized to oligonucleotides on the microarray following tagmentation and end-repairing. Unhybridized DNA fragments are washed away and the desired DNA molecules comprising one or more loci of interest are eluted. The DNA molecules comprising one or more loci of interest are then amplified using PCR (see, e.g., Turner et al., Methods for Genomic Partitioning, Annu Rev Genom Hum Genet., 10: 30-35 (2009); Mertes et al., Targeted Enrichment of Genomic DNA Regions for Next-Generation Sequencing, Brief Funct Genomics, 10: 374-86 (2011)).

To capture the DNA molecules comprising one or more loci of interest using in-solution capture, a pool of custom oligonucleotides (probes, DNA or RNA) is synthesized and hybridized in solution to the DNA molecules comprising one or more loci of interest. The probes selectively hybridize to the DNA molecules comprising one or more loci of interest after which the probe-DNA fragments of interest complex can be pulled down and washed to clear excess material. The probe-DNA fragments are then removed and the DNA molecules comprising one or more loci of interest can be sequenced allowing for selective DNA sequencing of genomic regions of interest (see, e.g., Kahvejian et al., What would You Do if You could Sequence Everything?, Nature Biotech., 26: 1125-33 (2008); Mamanova, Target-enrichment Strategies for Next Generation Sequencing, Nature Methods, 7: 111-18 (2010)). Agilent and NimbleGen are two exemplary in-solution target enrichment technologies.

In some embodiments, the probes, e.g., biotinylated hybridization probes would be target specific depending on the DNA methylation sites of interest. For example, the hybridization probes can be probes tested and validated to report on the most important and widely used human DNA methylation clocks (e.g., Human Clocks Mix; Horvath's Multi-tissue clock, Horvath, DNA Methylation Age of Human Tissues and Cell Types, Genome Biol.; 14:R115 (2013); Hannum's Blood Clock, Hannum et al., Genome-wide Methylation Profiles Reveal Quantitative Views of Human Aging Rates, Mol Cell. 49(2): 359-67 (2013); or Levine's PhenoAge clock, Levine et al., An Epigenetic Biomaker of Aging for Lifespan and Healthspan, Aging, 10(4): 573-91 (2018)); Cancer Clock, Zheng et al., Association of Epigenetic Clock with Consensus Molecular Subtypes and Overall Survival of Colorectal Cancer). Another example would be a probe set for rDNA methylation clock probes, that would capture/enrich for rDNA from human/mouse/rat and possible other mammals. There are many other possible combinations and applications of probe sets for DNA methylation-based biomarkers.

In some embodiments, the probes, e.g., biotinylated probes, include probes that are designed to enrich a DNA molecule comprising one or more loci of interest for determining a development status, e.g., aging, or a disorder or a disease, e.g., cancer, that is associated with certain DNA methylation status. For example, the probe may include oligonucleotide DNA or RNA that specifically or preferentially hybridizes with regions on the DNA comprising one or more loci of interest, the methylation of which is associated with a disease, e.g., cancer or aging. Exemplary diseases or disorders that exhibit characteristic DNA methylation status are disclosed elsewhere herein.

In certain embodiments, the DNA molecules comprising one or more loci of interest are enriched using in-solution enrichment strategy. The probe-DNA fragment complexes can be pulled down using any methods known in the art. In some embodiments, the probe is biotinylated DNA or RNA and the probe-DNA fragment complexes are pulled down via biotin-avidin interaction (see, e.g., Welcher et al., Nucleic Acids Research, 14: 10027-44 (1986).

In some embodiments, the probe-DNA fragment may be pulled down using a CRISPR/Cas9 mediated method. An example of CRISPR/Cas9 mediated pull down method is described in Xu et al. (CRISPR-assisted targeted enrichment-sequencing (CATE-seq), available at https://doi.org/10.1101/672816, incorporated herein by reference). After the pull-down, the probe may be removed. In some embodiments, the enrichment results in a single stranded DNA molecule, which comprises one strand of the double stranded DNA molecule comprising one or more loci of interest.

The method may further comprise converting the unmethylated cytosine in the end repaired double stranded DNA molecule or the enriched DNA comprising one or more loci of interest to uracil, thereby generating a cytosine-converted DNA molecule comprising one or more loci of interest. Any methods that convert the unmethylated cytosine can be used. For example, Williams et al. disclose a few methods for converting unmethylated cytosine (Williams et al., Enzymatic Methyl-seq: The Next Generation of Methylome Analysis, available at www.neb.com/tools-and-resources/feature-articles/enzymatic-methyl-seq-the-next-generation-of-methylome-analysis). In some embodiments, the treatment is bisulfite treatment.

The method may further comprise amplifying the cytosine-converted DNA molecules comprising one or more loci of interest, thereby generating an amplified double stranded DNA molecule comprising one or more loci of interest. For example, PCR may be used to amplify the cytosine-converted DNA molecules. The cytosine-converted DNA molecules as the PCR template have the general structure as follows
5′-the first adaptor-the first barcode-the first end sequence-cytosine-converted DNA molecule-the second end sequence-the second adaptor-3′

In some embodiments, additional sequences/components are added to the cytosine-converted DNA fragments. In some embodiments, the additional components are added to the cytosine-converted DNA fragments by ligation. In some embodiments, the additional components are incorporated to the cytosine-converted DNA fragments during the PCR amplification of the cytosine-converted DNA fragments.

To incorporate additional components to the converted DNA fragment by PCR, primers are designed and prepared to operably link the additional components to the 5′ terminal of the adaptor. In some embodiments, the two primers for amplifying the cytosine-converted DNA fragments have the following structure:
Forward Primer: 5′-the first universal primer-the first sequencing primer-the first adaptor-3′
Reverse Primer: 5′-the second universal primer-the second barcode-the second adaptor-3′

In some embodiments, the forward primer is a polynucleotide having the sequence of 5′-AATGATACGGCGACCACCGAAAGCAGTGGTATCAACGCAGATCTGGGTGGAGGG TGGTGGGTGGAGGGTGG-3′ (SEQ ID NO: 8), or a variant thereof.

In some embodiments, the forward primer is a polynucleotide having the sequence of 5′-AATGATACGGCGACCACCGAGATCTACACNNNNNNNNTCGTCGGCAGCGTC-3′ (SEQ ID NO: 18), or a variant thereof.

In some embodiments, the reverse primer is a polynucleotide having the sequence of 5′-CAAGCAGAAGACGGCATACGAGATNNNNNNNNGTCTCGTGGGCTCGGAGATGT-3′ (SEQ ID NO: 9), or a variant thereof.

In some embodiments, the reverse primer is a polynucleotide having the sequence of 5′-CAAGCAGAAGACGGCATACGAGATNNNNNNNNAAGCAGTGGTATCAACGCAGA TCTGGGTGGAGGGTGG-3′ (SEQ ID NO: 19), or a variant thereof.

Accordingly, after the amplification, the amplified double stranded DNA molecules have the structures as follows:
5′-the first universal primer-the first sequencing primer-the first adaptor-the first barcode-the first end sequence-the DNA molecules comprising one or more loci of interest-the second end sequence-the second adaptor-the second barcode-the second universal primer-3′

In certain embodiment, if the second adaptor in the above illustrations does not comprise a cytosine, a second sequencing primer may be added between the second adaptor and the second barcode.

The universal primers may be primers that are suitable for use in a next generation sequencing platform. In some embodiments, the first and the second universal primers are the primers in a commercially available next generation sequencing platform, e.g., the Illumina Nextera platform. Accordingly, one of ordinary skill in the art can readily choose the universal primers based on the sequencing platform to be used. In some embodiments, the first universal primer is the Illumina P5 primer having the sequence 5′-AAT GAT ACG GCG ACC ACC GA-3′ (SEQ ID NO: 10), or a variant thereof. In some embodiments, the second universal primer is the Illumina P7 primer having the sequence 5′-CAA GCA GAA GAC GGC ATA CGA GAT-3′ (SEQ ID NO: 11), or a variant thereof.

The first sequencing primer may be used as the primer for sequencing. The first sequencing primer may have a length that is suitable for sequencing. In some embodiments, the first sequencing primer has a length between 15 nt to 50 nt. In some embodiments, the first sequencing primer is a primer having the sequence 5′-AAG CAG TGG TAT CAA CGC AGA TCT GGG TGG AGG GTG G-3′ (SEQ ID NO: 12), or a variant thereof.

In some embodiments, the second barcode has a length between 4 nt and 15 nt. In some embodiments, the second barcode has a length of 8 nt, having a sequence of NNNNNNNN.

### C. Sequencing of the Converted DNA Molecules

The nucleotide sequences of the converted and amplified DNA molecules are determined. In certain embodiments, the converted and amplified DNA molecules are sequenced using next generation sequencing. Any suitable second generation sequencing method can be used to determine the nucleotide sequence of the converted and amplified DNA molecule. Exemplary next generation sequencing methods known in the art include, but are not limited to, sequencing-by-synthesis or sequencing-by-ligation platforms currently employed by Illumina, Life Technologies, and Roche, and nanopore sequencing methods or electronic-detection based methods such as Ion Torrent technology commercialized by Life Technologies. In some embodiments, the converted and amplified DNA molecules are sequenced using the Illumina Nextera platform.

The compositions and methods according to the present invention are suitable for multiplex sequencing based upon, at least partly, on the addition of two barcodes to the cytosine-converted and amplified double stranded DNA molecule. Optionally, one or more additional barcodes can be further added. In some embodiments, two or more libraries are pooled and sequenced. To distinguish the cytosine-converted and amplified double stranded DNA molecule from different libraries, the cytosine-converted and amplified double stranded DNA molecule within each library are tagged with unique barcode(s). In some embodiments, two barcodes that are located at the 5′ and 3′ terminal of the cytosine-converted and amplified double stranded DNA molecule molecules are library specific.

The nucleotide sequences of the cytosine-converted and amplified double stranded DNA molecule are compared to the nucleotide sequence of the target nucleotide sequence to determine the number and/or the location of the methylated cytosine on the DNA fragments.

### D. Exemplary Methods for Tagmentation-Based Indexing for MEthylation Sequencing (TIME-Seq) Library Preparation

In one aspect, the present invention provides a method for constructing a sequencing library for determining the methylation status of one or more loci of interest. The method includes fragmenting genomic DNA comprising one or more loci of interest to generate a plurality of double strand DNA molecules, wherein at least one of the plurality of double stranded DNA molecules comprises the one or more loci of interest; and preparing the plurality of double stranded DNA molecules comprising the one or more loci of interest according to any of the methods described herein, thereby generating a sequencing library for determining the methylation status of one or more loci of interest.

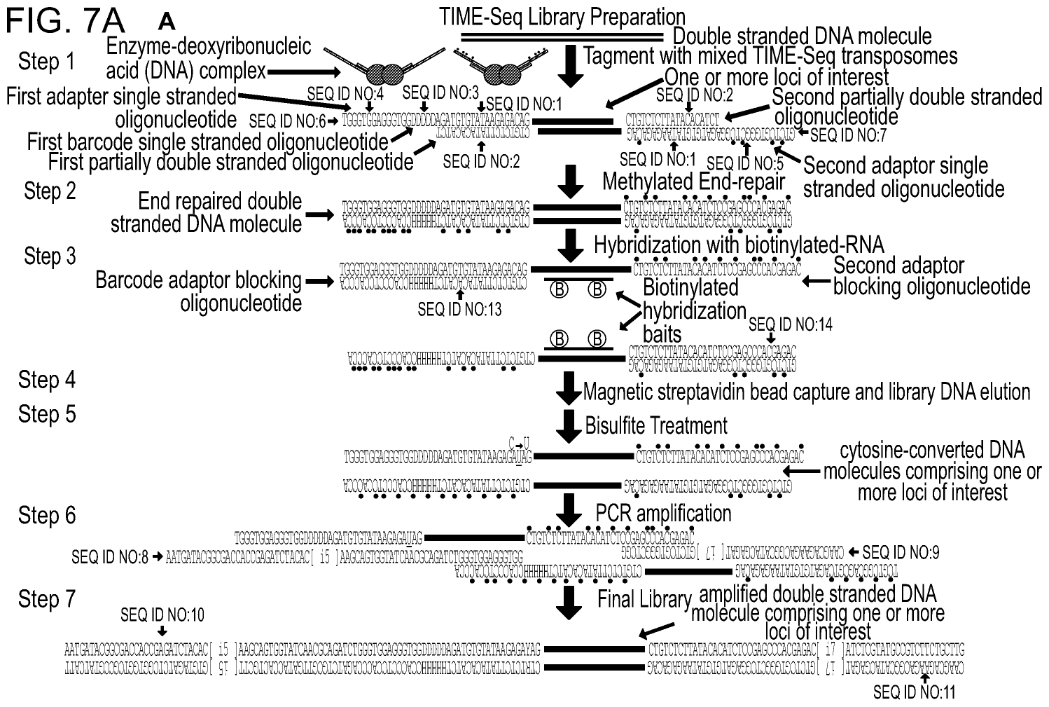

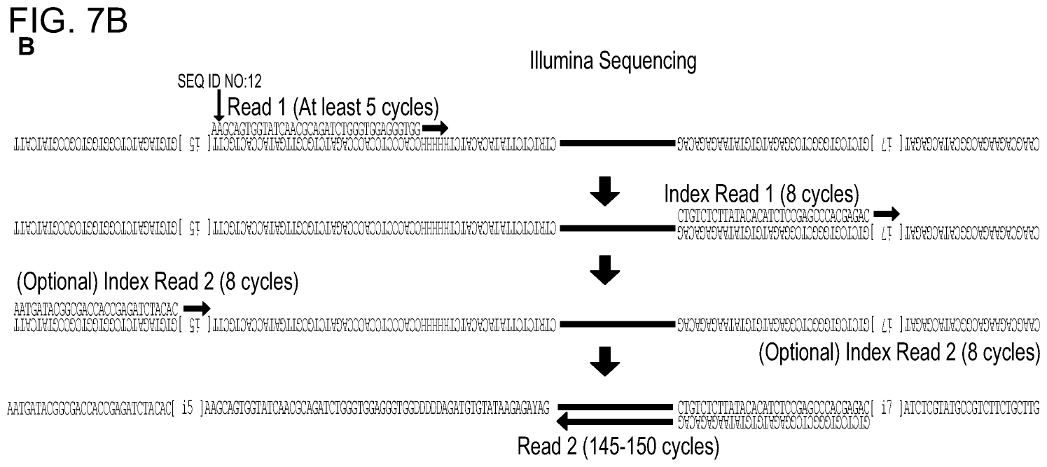

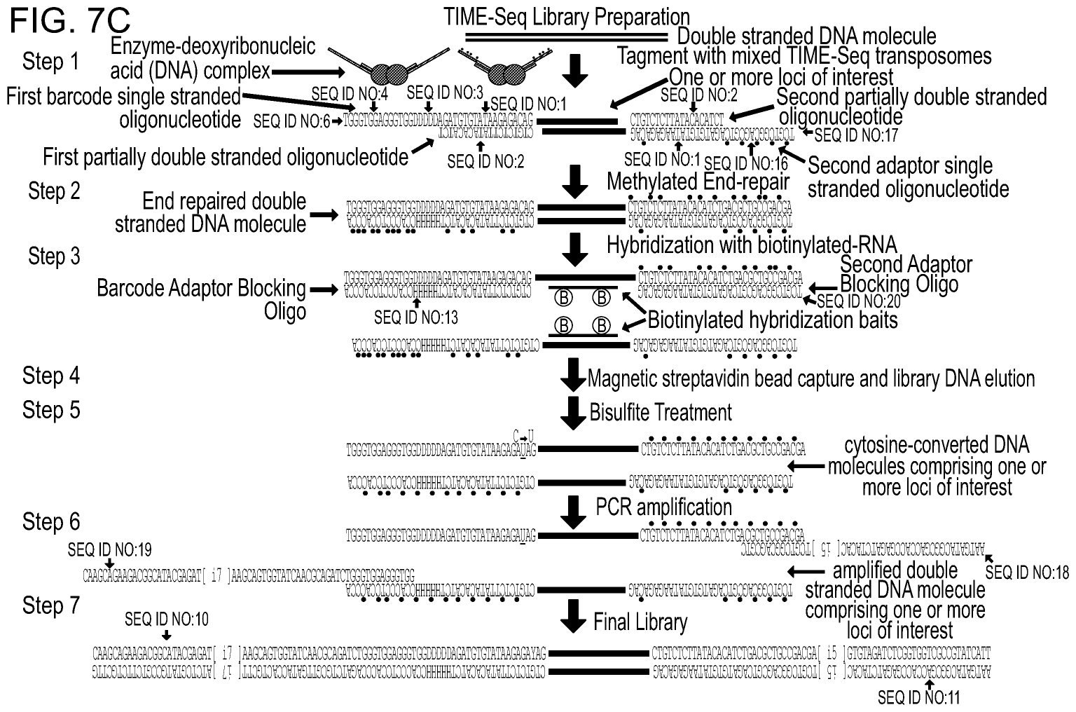

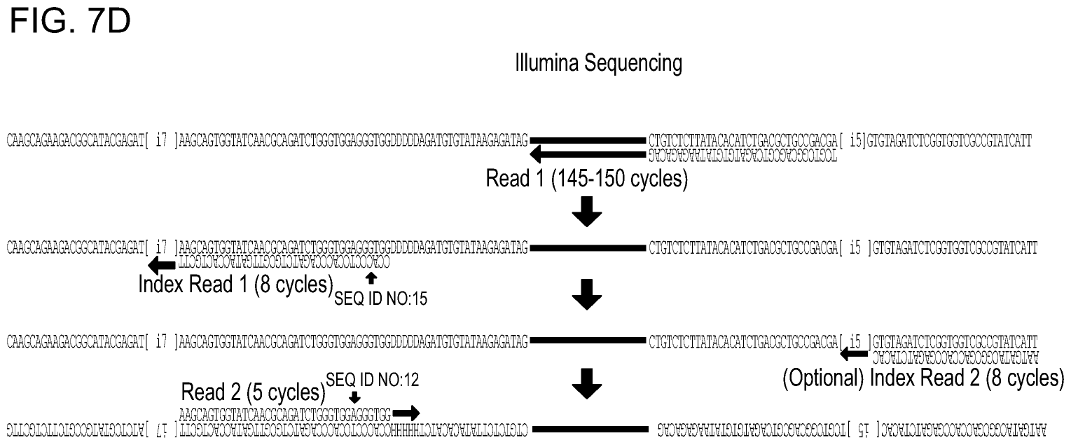

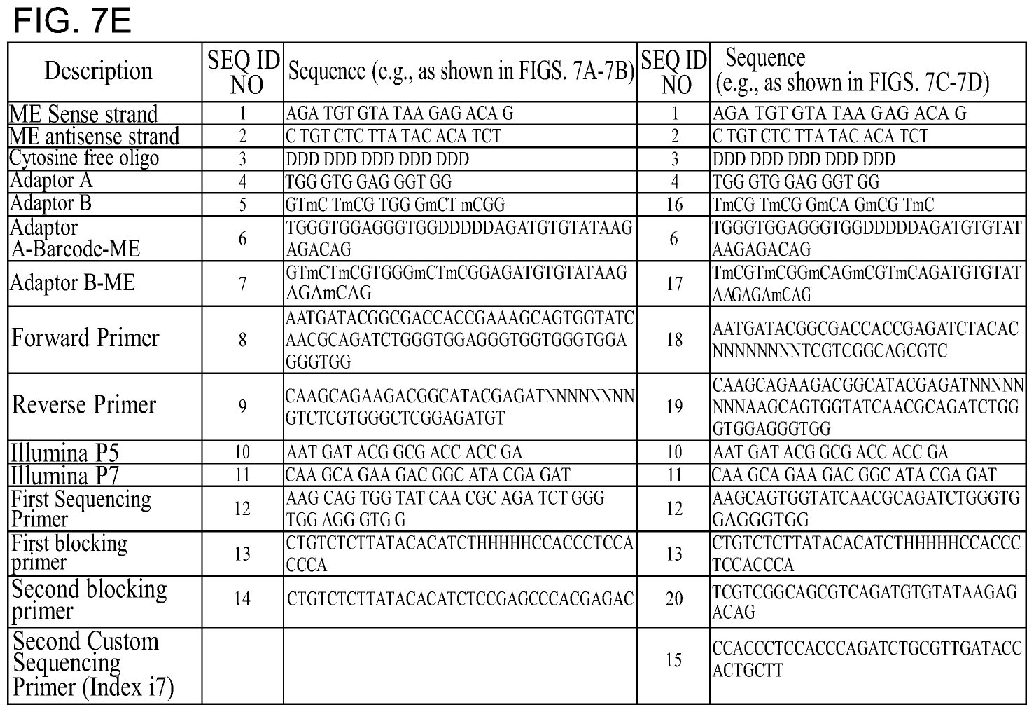

In certain embodiments, the present invention provides a method for constructing a sequencing library for determining the methylation status of one or more loci of interest as set forth in FIGS. 7A-7B and/or FIGS. 7C-7D.

In certain embodiments, the present invention provides a method for constructing a sequencing library for determining the methylation status of one or more loci of interest which comprise the use of an oligonucleotide, or a combination of oligonucleotides, as set forth in Table 8 or FIG. 7E.

In certain embodiments, the present invention provides a method for constructing a sequencing library for determining the methylation status of one or more loci of interest as set forth in FIGS. 7A-7B.

In certain embodiments, the present invention provides a method for constructing a sequencing library for determining the methylation status of one or more loci of interest as set forth in FIGS. 7C-7D.

In certain embodiments, the present invention provides a method for sequencing as set forth in FIG. 7B and/or FIG. 7D. Without being bound by theory, the sequencing method as set forth in FIG. 7B and/or FIG. 7D may be interchangeably used in combination with any suitable method described herein, such as a method as set forth in FIG. 7A and/or FIG. 7C. In certain embodiments, the method for sequencing comprises the use of an oligonucleotide, or a combination of oligonucleotides, as set forth in Table 8 or FIG. 7E.

FIGS. 7A-7B and FIGS. 7C-7D depict a graphical illustration of exemplary TIME-Seq library preparation and sequencing methods, and FIG. 7E and Table 8 depict exemplary oligonucleotides which may be used according to the methods described herein. In particular, FIGS. 7A-7B and FIGS. 7C-7D show an exemplary method for assembling an enzyme-deoxyribonucleic acid (DNA) complex for use in preparing a double stranded DNA molecule comprising one or more loci of interest for determining the methylation status of the one or more loci of interest therein, comprising, Exemplary Steps 1-7.

Exemplary Step 1 shows contacting an enzyme (e.g., a transposase) with a first partially double stranded oligonucleotide (e.g., SEQ ID NO: 6) comprising a first adaptor single stranded oligonucleotide (e.g., SEQ ID NO: 4) and a first barcode single stranded oligonucleotide (e.g., SEQ ID NO: 3), wherein the first adaptor oligonucleotide and the first barcode oligonucleotide are operably linked in the order, from 5′ to 3′, the first adaptor-the first barcode, and a second partially double stranded oligonucleotide (e.g., SEQ ID NO: 7 or SEQ ID NO: 17) comprising a second adaptor single stranded oligonucleotide (e.g., SEQ ID NO: 5 or SEQ ID NO: 16), wherein the enzyme is capable of operably linking the first and the second partially double stranded oligonucleotides to the double stranded DNA molecule comprising one or more loci of interest, thereby preparing the enzyme-DNA complex (e.g., a transposome). Notably, the first adaptor and the first barcode do not comprise a cytosine; the second adaptor does not comprise a cytosine or the cytosine thereon is methylated; and the nucleotide sequence of the first adaptor and the second adaptor are different. Enzymes are depicted as filled circles; and methylated cytosine is shown as a dot (●). This step differs from the Mulqueen method (Mulqueen, Pokholok et al. 2018), for example, in that two separate adaptors, SEQ ID NO: 4 and SEQ ID NO: 5 (FIG. 7A) or SEQ ID NO: 4 and SEQ ID NO: 16 (FIG. 7C), that are resistant to bisulfite conversion are transposed to barcode each sample and provides the following advantages which cannot be achieved by the Mulqueen method including, for example, compatibility with immediate end-repair to create DNA fragments compatible with paired-end sequencing and compatibility with efficient hybridization enrichment of target DNA.

In certain embodiments, SEQ ID NO: 5, which refers to methylated Read 2 Adaptor (i.e., Nextera Adaptor B: GTmC TmCG TGG GmCT mCGG), may be used interchangeably with SEQ ID NO: 16, which refers to a methylated Read 1 Adaptor (i.e., Nextera Adaptor A: TmCG TmCG GmCA GmCG TmC). Accordingly, in some embodiments, the sequence of the primers to amplify DNA may also be different (see, e.g., Step 6). However, in certain embodiments, regardless of whether SEQ ID NO: 5 or SEQ ID NO: 16 is used, the second adaptor is the barcoded cytosine depleted adaptor. In certain embodiments, use of SEQ ID NO: 16 instead of SEQ ID NO: 5 according to the methods described herein may provide for higher read quality of the sequenced DNA in Read 1 as opposed to Read 2, for example, when sequenced using a 150 cycle kit.

Exemplary Step 2 shows repairing the ends of double stranded DNA molecule comprising one or more loci of interest operably linked to the first partially double stranded oligonucleotide and the second partially double stranded oligonucleotide using methylated cytosine, thereby generating an end repaired double stranded DNA comprising one or more loci of interest. This step differs from the Mulqueen method, for example, in that the transposed adaptors are compatible with immediate end-repair of pooled barcoded DNA fragments and provides the following advantages which cannot be achieved by the Mulqueen method including, for example, efficient hybridization enrichment of target DNA with two bisulfite resistant adaptors that are compatible with paired-end sequencing of bisulfite converted DNA.

Exemplary Step 3 shows an in-solution target enrichment method utilizing biotinylated RNA bait hybridization. This step differs from the Mulqueen method in that DNA containing two bisulfite resistant adaptors are annealed by complementary blocking oligonucleotides, for example, SEQ ID NO: 13 and SEQ ID NO: 14 (FIG. 7A), or SEQ ID NO: 13 and SEQ ID NO: 20 (FIG. 7C), which prevent the hybridization of adaptor DNA to each other while target DNA is bound by pre-designed biotinylated hybridization baits that can be captured by streptavidin affinity beads, which provides the following advantages which cannot be achieved by the Mulqueen method including, for example, the potential to enrich for specific DNA of interest.

Exemplary Step 4 (not illustrated) includes the capture of the complex formed by the hybridization of the DNA and bait oligo, for example, using streptavidin magnetic beads. This step differs from the Mulqueen method in that target DNA is enriched via streptavidin-biotin affinity capture of biotin-RNA bound to DNA and provides the following advantages which cannot be achieved by the Mulqueen method including, for example, enrichment of target DNA to increase the fraction of reads sequenced that contain target DNA.

Exemplary Step 5 shows the process of converting the unmethylated cytosine in the end repaired double stranded DNA molecule comprising one or more loci of interest or the enriched DNA molecule comprising one or more loci of interest to uracil, thereby generating a cytosine-converted DNA molecule comprising one or more loci of interest. The unmethylated cytosine may be converted into uracil via bisulfite treatment. This step differs from the Mulqueen method in that DNA molecules with two different bisulfite conversion resistant adaptors are subjected to bisulfite conversion and provides the following advantages which cannot be achieved by the Mulqueen method including, for example, amplification of bisulfite converted DNA in the next step via polymerase chain reaction. In contrast, in the Mulqueen method, DNA with only one adaptor is bisulfite converted and then linear amplification is required to add a second adaptor before polymerase chain reaction (PCR) can be used to amplify DNA to a molarity that is desirable for sequencing.

Exemplary Step 6 shows the process of amplifying the cytosine-converted DNA molecule comprising one or more loci of interest, thereby generating an amplified double stranded DNA molecule comprising one or more loci of interest. The amplification may comprise polymerase chain reaction (PCR). This step differs from the Mulqueen method in that DNA is amplified immediately after bisulfite conversion and DNA clean-up, and provides the following advantages which cannot be achieved by the Mulqueen method including, for example, avoiding linear amplification and random priming that has been shown to decrease insert DNA fragment length (Miura et al, NAR, 2019), and which is not ideal for hybridization enrichment methods in which longer DNA inserts have an increased chance of annealing to and staying annealed to hybridization baits.

Exemplary Step 7 shows the process of operably linking a double stranded oligonucleotide comprising a first universal primer and a first sequencing primer to the first adaptor and a second double stranded oligonucleotide comprising a second universal primer and a second barcode to the second adaptor, wherein the nucleotide sequence of the first universal primer and the second universal primer is different. The cytosine converted DNA molecule may include one or more loci of interest, the first universal primer, and the first sequencing primer that are operably linked in the followed order: 5′-the first universal primer-the first sequencing primer-the cytosine converted DNA-3′.

In certain embodiments, the present invention provides a method for constructing a sequencing library for determining the methylation status of one or more loci of interest as set forth in FIGS. 7A-7B and/or FIGS. 7C-7D, which further comprises a sequencing step, e.g., as set forth in FIG. 7B and/or FIG. 7D.

In certain embodiments, the sequencing may comprises shallow sequencing (i.e., coverage of 1-2 reads per CpG).

In certain embodiments, the sequencing comprises Illumina sequencing, for example, in which four sequencing primers are used for a paired-end sequencing and dual indexed library. In certain embodiments, in which the method comprises sequencing, e.g., Illumina sequencing, the following reads may be sequenced: (1) Read 1, (2) Index Read 1 (typically referred to as i7); (3) Index Read 2 (typically referred to as i5); and (4) Read 2.

In certain embodiments, the sequencing, e.g., Illumina sequencing, comprises single indexing. In certain embodiments, in which the method comprises single indexing, the following reads may be sequenced: (1) Read 1 (e.g., using a custom primer, e.g., a primer comprising the sequence of SEQ ID NO: 12); (2) Index Read 1 (typically referred to as i7); and (4) Read 2.

In certain embodiments, the sequencing, e.g., Illumina sequencing, comprises dual indexing. In certain embodiments, in which the method comprises dual indexing, the following reads may be sequenced: (1) Read 1; (2) Index Read 1 (typically referred to as i7) (e.g., using a primer comprising the sequence of SEQ ID NO: 15); (3) Index Read 2 (typically referred to as i5); and (4) Read 2 (e.g., using a primer comprising the sequence of SEQ ID NO: 12).

In certain embodiments, the sequencing, e.g., Illumina sequencing, comprises dual indexing. In certain embodiments, in which the method comprises dual indexing, the following reads may be sequenced: (1) Read 1 (e.g., using a primer comprising the sequence of SEQ ID NO: 12); (2) Index Read 1 (typically referred to as i7); (3) Index Read 2 (typically referred to as i5) (e.g., using a primer comprising the sequence of SEQ ID NO: 15); and (4) Read 2. In certain embodiments, the sequencing is performed on a machine that does not have a graphed primer, for example, on a MiSeq machine.

In some embodiments, the methods described herein comprise determining the methylation status of single cells. In some embodiments, the methods described herein do not comprise determining the methylation status of single cells. In some embodiments, the methods described herein comprise determining the methylation status of a population of cells.

In some embodiments, the methods described herein comprise determining the methylation status of individual nuclei, e.g., isolated from a plurality of cells. In some embodiments, the methods described herein do not comprise determining the methylation status of individual nuclei, e.g., isolated from a plurality of cells.

In some embodiments, the methods described herein comprise isolating nuclei from a plurality of cells. In some embodiments, the methods described herein do not comprise isolating nuclei from a plurality of cells.

In some embodiments, the methods described herein do not comprise subjecting isolated nuclei to a chemical treatment to generating nucleosome-depleted nuclei, while maintaining integrity of the isolated nuclei.

In some embodiments, the methods described herein comprise purifying nucleic acids, e.g., DNA, from cells and/or nuclei. In some embodiments, the nucleic acids, e.g., DNA, is substantially free of other cellular components, such as proteins, lipids, sugars, etc.

In certain embodiments, the methods described herein do not comprise fragmenting nucleic acids in subsets of nucleosome-depleted nuclei into a plurality of nucleic acid fragments and incorporating only a single barcode sequence into at least one strand of the nucleic acid fragments to generate barcoded nuclei. Without being bound by theory, the addition of two different adaptors, as described herein, enables immediate PCR amplification of DNA after target enrichment and bisulfite conversion, which is an efficient order of steps for certain embodiments of the methods described herein which obviates the need for re-pooling of nucleic acids after transposase tagmentation. In some embodiments, the methods described herein do not comprise separating or redistributing pooled nucleic acids into separate compartments or wells, for example, after transposase tagmentation.

In certain embodiments, the methods described herein do not use linear amplification and random priming after bisulfite conversion to add a second adaptor. Instead, in certain embodiments, the methods described herein incorporate a second adaptor at the same time as the first adaptor, for example, by transposase tagmentation. In certain embodiments, the methods described herein, do not use random priming, for example, to add a second adaptor.

E. Exemplary Methods for Highly Accurate Age Prediction from Shallow Sequencing of TIME-Seq Libraries

In one aspect, the present invention provides a method for predicting age from shallow sequencing of TIME-Seq libraries. In certain embodiments, a method for predicting age from shallow sequencing of TIME-Seq libraries may comprise TIME-Seq libraries which are sequenced with 5 to 30 thousand reads per sample. In certain embodiments, a method for predicting age from shallow sequencing of TIME-Seq libraries may comprises creating a sparse methylation matrix in which most CpGs may be covered by only about 1 to about 2 reads. In certain embodiments, a method for predicting age from shallow sequencing of TIME-Seq libraries may comprises creating a sparse methylation matrix most samples comprise very few overlapping CpGs in a pairwise comparison.

In certain embodiments, a method for predicting age from shallow sequencing of TIME-Seq libraries may comprise applying a modified version of the scAge algorithm to the TIME-Seq data. Without being bound by theory, in certain embodiments, the methods described herein comprising applying a scAge algorithm, or modified version thereof, create linear models to predict methylation from age using a previously described bulk sequenced dataset and then taking a maximum likelihood approach to predict age from the shallow sequencing data. Due to the targeted nature of TIME-Seq libraries, it is especially amenable to this approach for age prediction since the CpGs that are covered in TIME-Seq libraries are highly overlapping with those included in the linear models when compared to non-enriched random DNA methylation sequencing such may be obtained in single cell methylation data.

In certain embodiments, a method for predicting age from shallow sequencing of TIME-Seq libraries may achieve age predictions from shallow sequencing of TIME-Seq data that are highly accurate, e.g., with correlations of about R=0.826 to about R=0.947, for example, with median absolute error of prediction of less than about 1 month, or between about 1 to about 4 months, or about 1, about 1, about 3, or about 4 months. In certain embodiments, age prediction results achieved according to a method described herein may be as accurate, or more accurate, as compared to a deeply sequenced clock (i.e., coverage of 50-100+ reads per CpG). Further, in certain embodiments, age prediction from shallow sequencing (i.e., coverage of 1-2 reads per CpG) according to a method described herein may be as accurate, or more accurate, as from bulk sequencing, using this method or any other suitable method.

### III. Kits of the Invention

Any of the compositions described herein may be comprised in a kit. In a non-limiting example, the kit comprises a partially double stranded oligonucleotide and an integrase complex, e.g., a transposome. In some embodiments, the kit comprises two partially double stranded oligonucleotide and integrase complexes, e.g., transposomes.

Preferably, the two partially double stranded oligonucleotide and integrase complexes comprise the same integrase. For example, two transposomes in a kit may both comprise hyperactive transposase Tn5.

In some embodiments, the kit comprises the components of the partially double stranded oligonucleotide and integrase complex, e.g., the transposome, separately. That is, the partially double stranded oligonucleotide is not assembled with the integrase, e.g., transposase. The partially double stranded oligonucleotide and the integrase, e.g., transposase, are assembled prior to the use.

In some embodiments, the kit of the present invention includes hyperactive transposase Tn5 and a partially double stranded oligonucleotide that comprises an ME end sequence as described herein.

The integrase-partially double stranded oligonucleotide complex, e.g., the transposome, may be provided in a format that is suitable for large scale application. For example, the transposome, or the components thereof, may be provided in 12 well strip, or 96 well, 384 well, or 1536 well plate.

The kit may further include reagents or instructions for use of the partially double stranded oligonucleotide and integrase, e.g., transposome. It may also include one or more buffers.

In certain embodiments, a kit may include an oligonucleotide as described herein, for example, as set forth in FIG. 7E and/or Table 8.

The kit may further include the compositions described herein for preparing the partially double stranded oligonucleotide and integrase complex, e.g., the transposome, and/or for preparing a library for sequencing. An exemplary kit may include the components as shown in Table 1 below.

TABLE 1

| Tagmentation and Library | | |
|---|---|---|
| Preparation | Target Enrichment | Amplification |
| The first and second partially | Blocking primers | Forward and |
| double stranded | | Reverse |
| oligonucleotide | | Primers |
| Purified Transposase Tn5 | Biotinylated | |
| | RNA/DNA | |
| | hybridization | |
| | probes | |
| 2XTD Buffer (20 mM Tris pH | | |
| 7.8, 10 mM MgCl₂, 20% DMF | | |
| [Dimethylformamide]) | | |
| STOP Buffer (100 mM MES | | |
| pH 5, 4.125M Guanidine | | |
| Thiocyanate, 25% | | |
| Isopropanol, 10 mM EDTA) | | |
| 10X dNTPS (5-methyl-dCTP, | | |
| dATP, dTTP, dGTP, each at | | |
| 10 mM) | | |

The blocking primers comprise the reverse complement of the adaptors. In some embodiments, the first blocking primer has the following structure:
5′-the anti-sense strand of the hyperactive mosaic end sequence-degenerate nucleotides-the reverse complement of the first adaptor-3′

The length of the degenerate nucleotides corresponds to that of the first barcode. For example, if the first barcode is 5 nucleotides in length, the degenerate nucleotides is 5 nucleotides in length as well.

In some embodiments, the second blocking primer has the following structure:
5′-the anti-sense strand of the hyperactive mosaic end sequence-degenerate nucleotides-the reverse complement of the second adaptor-3′

In some embodiments, the probes, e.g., biotinylated probes, include probes that are designed to enrich a DNA molecule comprising one or more loci of interest for determining a development status, e.g., aging, or a disorder or a disease, e.g., cancer, that is associated with certain DNA methylation status. For example, the probe may include oligonucleotide DNA or RNA that specifically or preferentially hybridizes with regions on the DNA comprising one or more loci of interest, the methylation of which is associated with a disease, e.g., cancer or aging. Exemplary diseases or disorders that exhibit characteristic DNA methylation status are disclosed elsewhere herein.

The kit may further include reagents or instructions for using the transposome or the components thereof. It may also include one or more buffers.

The components of the kits may be packaged either in aqueous media or in lyophilized form. The container means of the kits will generally include at least one vial, test tube, flask, bottle, or other container means, into which a component may be placed, and preferably, suitably aliquoted. Where there is more than one component in the kit (e.g., labeling reagent and label may be packaged together), the kit also will generally contain a second, third or other additional container into which the additional components may be separately placed. The kits may also comprise a second container means for containing a buffer. However, various combinations of components may be comprised in a vial. The kits of the present invention also will typically include a means for containing the compositions of the invention, e.g., the transposome, and any other reagent containers in close confinement for commercial sale.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

### IV. Detection of the DNA Methylation as a Biomarker Using the Methods of the Invention

DNA methylation status is associated with human development status and diseases or disorders. A subject, e.g., a human, in a particular development stage, e.g., aging, or having a disease or disorder, e.g., cancer, may have characteristic DNA methylation status on a genomic DNA molecule comprising one or more loci of interest. Accordingly, the methylation status on a DNA molecule comprising one or more loci of interest may serve as a biomarker for development status, or disease or disorders.

The low cost and compatibility with sample multiplexing render the methods of the invention suitable for large scale and/or high-throughput determination of DNA methylation status. Accordingly, in some embodiments, the present invention features methods for determining the DNA methylation status of one or more subjects in need thereof using the methods disclosed herein. The determination of methylation status can be used for various purposes, including, but not limited to, measurement of aging status, diagnosing a disease or disorder, e.g., cancer, determining the prognosis of a disease or disorder, e.g., cancer, and/or evaluating the efficacy of a treatment of a disease or disorder, e.g., cancer chemotherapy.

In some embodiments, the methods of the present invention include comparing the DNA methylation status of a DNA molecule comprising one or more loci of interest to a reference DNA methylation status. A “reference DNA methylation status,” as used herein, refers to a baseline DNA methylation status for evaluating the DNA methylation status of a DNA molecule comprising one or more loci of interest. For example, the reference DNA methylation status may be the mean DNA methylation status on a DNA molecule comprising one or more loci of interest present in a population. Thus, a subject suffering a disease, e.g., cancer, may have a DNA methylation status on a DNA molecule comprising one of more loci of interest deviating from the reference DNA methylation status. The reference DNA methylation status may also refer to the DNA methylation status of the DNA molecule comprising one or more loci of interest from a healthy tissue in a subject. Thus, the subject's DNA methylation status of a diseased tissue, e.g., cancer tissue, may be evaluated by comparing to the reference DNA methylation status. The reference DNA methylation status may also refer to the DNA methylation status on a DNA molecule comprising one or more loci of interest before a treatment, e.g., chemotherapy of a cancer. Thus, the DNA methylation status on a DNA molecule comprising one or more loci of interest after the treatment may be compared to the reference DNA methylation status to evaluate the efficacy of the treatment.

Exemplary diseases or disorders that are associated with characteristic DNA methylation status include, but are not limited to cancers, autoimmune diseases, metabolic disorders, neurological disorders, and viral infections. It was known in the art that the DNA methylation status on a DNA molecule comprising one or more loci of interest is associated with development stage and/or diseases or disorders. See, e.g., Jin & Liu, DNA Methylation in Human Diseases, Genes & Diseases, 5:1-8 (2018).

Exemplary cancers include, but are not limited to, colon and rectal cancer, breast cancer, liver cancer, lung cancer, bladder cancer, Wilms cancer, ovarian cancer, esophageal cancer, prostate cancer, head and neck cancer, bone cancer, kidney cancer, lip and oral cancer, non-small cell lung cancer, small cell lung cancer, pancreatic cancer, thyroid cancer, endometrial cancer, central nervous system cancer, melanoma, non-melanoma skin cancer, mesothelioma, hepatocellular carcinoma, glioblastoma, squamous cell lung cancer, thyroid carcinoma, and leukemia.

Exemplary autoimmune diseases include, but are not limited to, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barre, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes (such as type I diabetes), Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, Wegener's Granulomatosis, and myasthenia gravis.

Exemplary metabolic disorders include, but are not limited to, diabetes, cardiovascular disease, metabolic syndrome, insulin-resistance, nonalcoholic steatohepatitis, non-alcoholic fatty liver disease, viral hepatitis, liver cirrhosis, liver fibrosis, diabetic retinopathy, diabetic neuropathy, diabetic nephropathy, beta cell depletion, insulin resistance in a patient with congenital adrenal hyperplasia treated with a glucocorticoid, dysmetabolism in peritoneal dialysis patients, reduced insulin secretion, improper distribution of brown fat cells and white fat cells, obesity, improper modulation of leptin levels, hyperglycemia, hyperlipidemia, and dyslipidemia.

Exemplary neurological disorders include, but are not limited to MLS (cerebellar ataxia), Huntington's disease, Alzheimer's disease (AD, for example familial AD and/or sporadic AD), dementia, age-related dementia, Parkinson's disease (PD), cerebral edema, amyotrophic lateral sclerosis (ALS), Pediatric Autoimmune Neuropsychiatric Disorders Associated with Streptococcal Infections (PANDAS), meningitis, hemorrhagic stroke, autism spectrum disorder (ASD), brain tumor, Down syndrome, multi-infarct dementia, status epilecticus, contusive injuries (e.g., spinal cord injury and head injury), viral infection induced neurodegeneration, (e.g., AIDS, encephalopathies), epilepsy, benign forgetfulness, closed head injury, sleep disorders, major depressive disorder, dysthymia, seasonal affective disorder, dementias, movement disorders, psychosis, alcoholism, post-traumatic stress disorder, and Rett syndrome.

Exemplary viral infections include, but are not limited to infection with hepatitis A, HIV, HTLV-1, HTLV-II, influenza A, influenza B, respiratory syncytial virus (RSV), herpes simplex virus types 1 and 2 (HSV), varicella zoster virus (VZV), cytomegalovirus (CMV), Epstein-Barr virus (EBV), human herpes virus type 6 (HHV6), human herpes virus type 7 (HHV-7), human herpes virus type 8 (HHV-8), human papilloma virus infection, rotavirus, adenovirus, SARS virus, poliovirus, encephalomyocarditis virus (EMCV), smallpox virus, picornaviruses, caliciviruses, nodaviruses, coronaviruses, arteri viruses, flaviviruses, and togaviruses.

The present invention is further illustrated by the following examples, which should not be construed as limiting. The entire contents of all of the references cited throughout this application are hereby expressly incorporated herein by reference.

### EXAMPLES

### Example 1. Design of Methods for Methylation Determination

An epigenetic mark called DNA cytosine methylation (DNAm) is a conserved epigenetic modification that influences expression of genes. This mark has been used by researchers in both academia and industry as a biomarker to determine the status of a cancer, the biological age of a blood or tissue sample, and for forensics. Even though the cost of DNA sequencing has fallen dramatically, the cost of obtaining a readout for a DNAm biomarker is still cost-prohibitive for most researchers working with dozens to thousands of samples. The most common methods can cost between $200-600 per sample. If the cost of the testing of a DNAm biomarker could be reduced 10- to 100-fold, it would be used routinely in medicine and research.

As described herein, a method has been developed for cost-effective and high-throughput bisulfite sequencing (BS) to assay DNA cytosine methylation (DNAm) at targeted sets of CpGs, which is compatible with multiplexing of samples and can be applied to drastically reduce the cost of assaying DNA methylation-based biomarkers in the fields of aging, cancer, and more. Currently, there are two assays which are predominately used to measure such biomarkers. For most human studies, Illumina microarray-based methylation chips (e.g. the Infinium MethylationEPIC Chip that measures DNA methylation at 850,000 CpGs) are used. These arrays are not compatible with sample multiplexing and cost upward of $280 per sample. In mouse studies, the most common method for methylation clock analysis is Reduced-Representation Bisulfite Sequencing (RRBS) (Meissner, A. et al. Reduced representation bisulfite sequencing for comparative high-resolution DNA methylation analysis. Nucleic acids research 33, 5868-5877, (2005); Gu, H. et al. Preparation of reduced representation bisulfite sequencing libraries for genome-scale DNA methylation profiling. Nature protocols 6, 468-481, (2011)). This protocol, while designed to increase sequencing coverage of CpG dense regions, nonetheless, has limited multiplexing capacity and suffers from substantial batch effects based on variable restriction enzyme digestion used to prepare libraries.

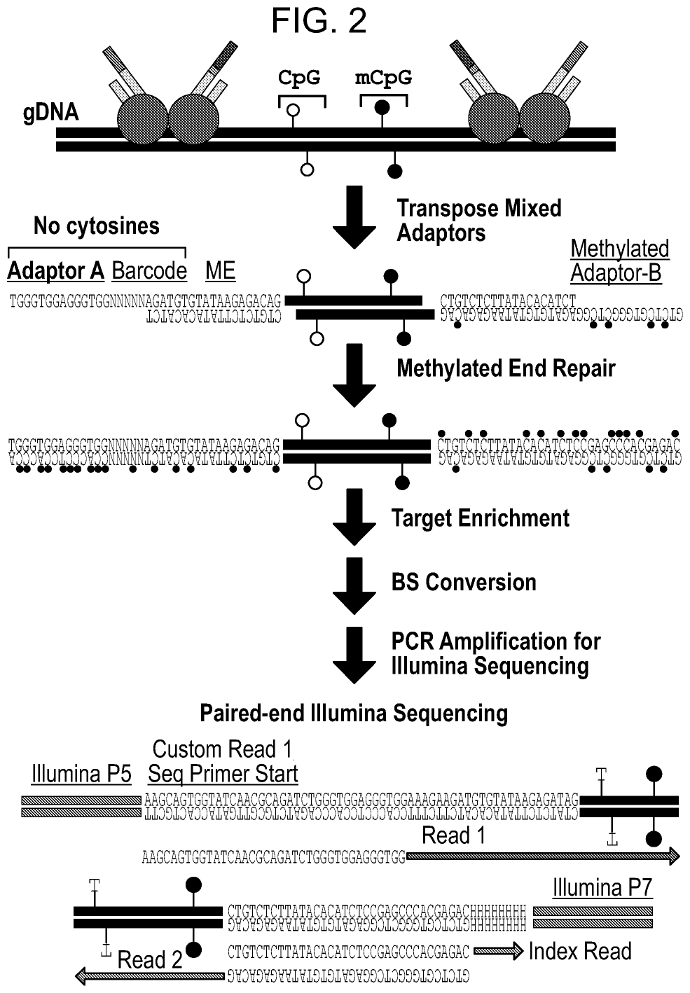

Here, a novel transposon-based library preparation and sequencing protocol were developed. The protocol is compatible with highly multiplexed bisulfite-sequencing (FIG. 2), and reduces the cost of targeted methylation sequencing 1-2 orders of magnitude as compared to currently available methods. The library preparation relies on incorporation of mixed sequencing adaptors/barcodes, or other oligonucleotides for sequencing with several key modifications and is compatible with sequencing on an Illumina MiSeq. More specifically, the methods includes a first partially double stranded oligonucleotide which contains a downstream barcode region and a first adaptor (Adaptor A as shown in FIG. 2) that are both completely devoid of cytosines (only A,T,G bases), while the second adaptor (Adaptor B in FIG. 2) is a primer used in a next generation sequencing platform, e.g., a primer used in a Nextera sequencing platform (Illumina), synthesized with methylated cytosines. After transposition, the samples were pooled to undergo end-repair with 5-methyl-dCTP in one tube, which protects the reverse strand of the adaptors from bisulfite conversion. Next, targeted enrichment protocols, such as in-solution biotinylated RNA bait hybridization (Gnirke, A. et al. Solution hybrid selection with ultra-long oligonucleotides for massively parallel targeted sequencing. Nature biotechnology 27, 182-189, doi:10.1038/nbt.1523 (2009)), are applied to the pool to increase sequencing depth at loci of interest. Finally, PCR amplification added P5 and P7 adaptors for Illumina sequencing while incorporating a first sequencing primer (Custom Read 1 Seq Primer in FIG. 2) start-site downstream of the P5 sequence.

In summary, these modifications allowed for immediate pooling of samples while protecting both adaptor sequences and their complementary strands from sequence change or modification during sodium bisulfite deamination of unmethylated cytosines to uracils. After bisulfite treatment, the pool can be enriched for target loci and PCR amplified for Illumina sequencing with pool-specific indices.

### Example 2. Library Preparation

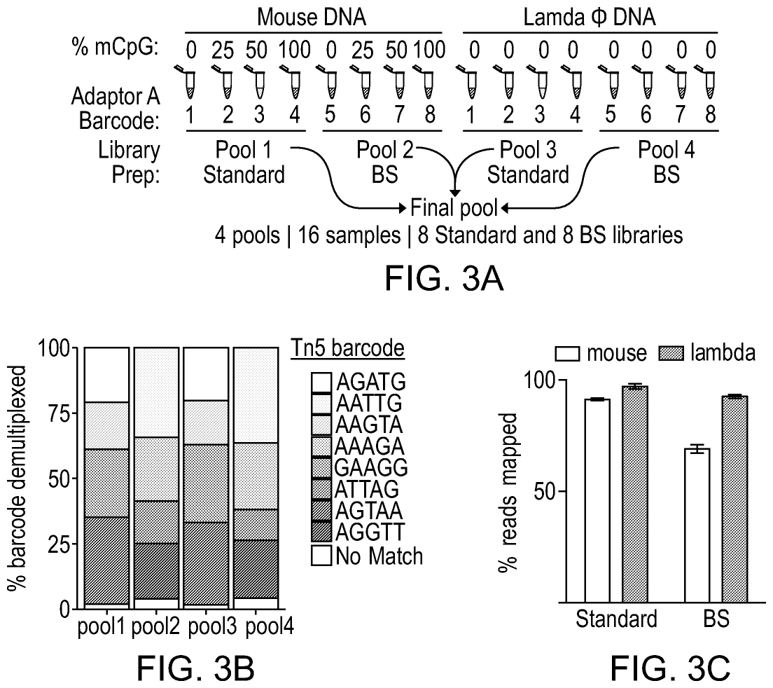

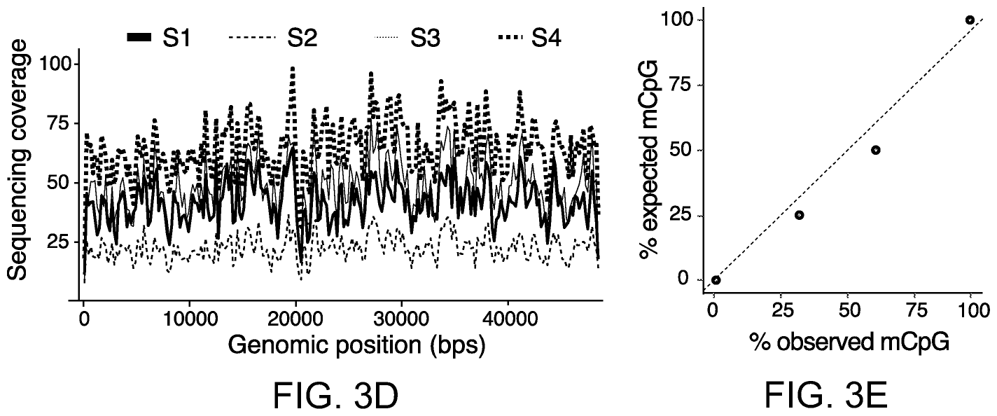

To test this multiplexing and sequencing strategy, a pilot experiment was performed on 16 DNA samples from mouse and lambda phage using both bisulfite conversion and standard library preparation protocols (FIG. 3A). The mouse DNA was derived from enzymatically methylated standards, representing approximately 0, 25, 50, and 100% CpG methylation. The lambda phage DNA, a common spike-in control for bisulfite sequencing (BS), was completely unmethylated. DNA was tagmented with home-made Tn5 (Picelli, S. et al. Tn5 transposase and tagmentation procedures for massively scaled sequencing projects. Genome research 24, 2033-2040, doi:10.1101/gr.177881.114 (2014)) and pooled for library preparation. Shallow sequencing (<1 million reads) was performed using an Illumina Mi-Seq and an analysis pipeline was implemented to demultiplex, trim, and map both standard and bisulfite converted reads, and extract DNA methylation information.

Sample libraries from each pool were demultiplexed efficiently based on the Tn5 barcode (FIG. 3B) and mapped at a high rate (FIG. 3C) to either the mouse or lambda genomes for both the standard and bisulfite-converted libraries. The bisulfite converted lambda DNA libraries showed considerable sequencing coverage across the 48 kilobase lambda genome (FIG. 3C) and a non-conversion rate of less than or equal to 0.4%. Bisulfite sequencing mouse DNA libraries had approximately the same non-conversion rate based on the non-CpG cytosines. The observed CpG methylation for mapped reads corresponds to the estimated CpG methylation for each standard (FIG. 3E).

### Example 3. Target Enrichment

To test the multiplexing and sequencing strategy, a proof-of-concept in-solution enrichment experiment was performed using biotinylated RNA-baits targeting the mouse blood DNA methylation clock, which is a robust biomarker of age based on the methylation level of 90 CpGs in the mouse genome (Petkovich, D. A. et al. Using DNA Methylation Profiling to Evaluate Biological Age and Longevity Interventions. Cell metabolism 25, 954-960.e956, doi:10.1016/j.cmet.2017.03.016 (2017)). Twelve (12) samples were tagmented with different barcoded, cytosine-depleted mixed adaptors and pooled for hybridization enrichment. After enrichment, the pool was amplified and sequenced on an Illumina Miseq using a micro V2 kit.

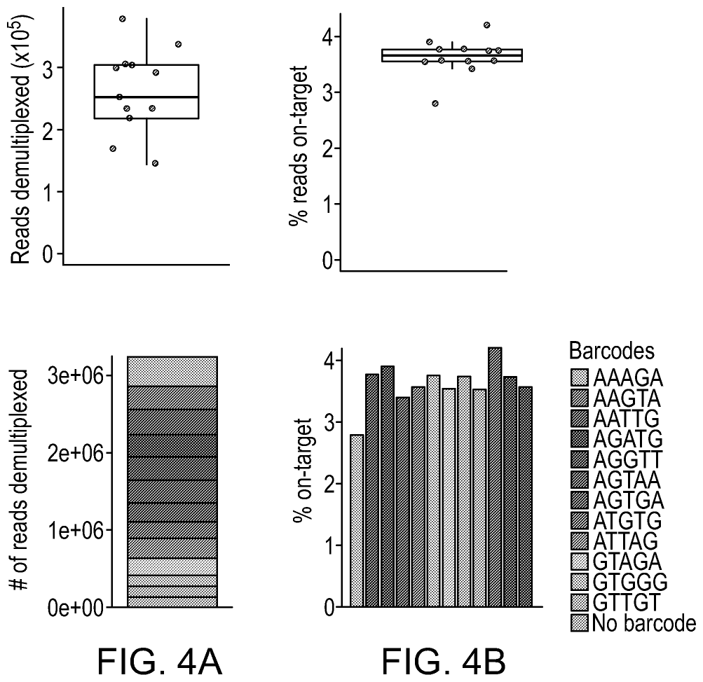

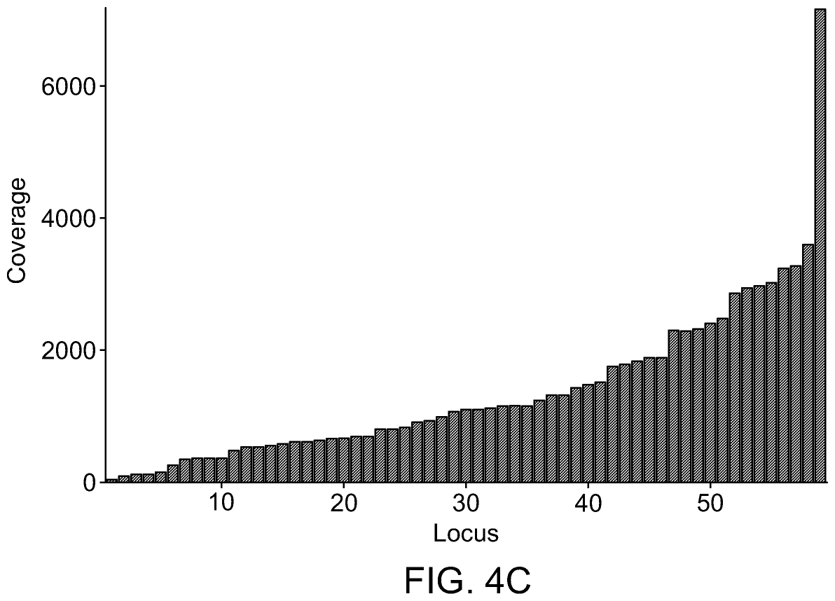

All samples efficiently demultiplexed based on the Tn5 Adaptor A barcode (FIG. 4A) and each sample had a substantial percentage of reads that mapped to the target loci (FIG. 4B). Coverage across all loci was roughly Poisson in distribution, with more than enough coverage across each locus to compute a DNA methylation biomarker readout. These results demonstrate that the novel library preparation and custom sequencing approach described herein is compatible with highly multiplexed bisulfite sequencing that can be combined with targeted sequence enrichment of DNA methylation-based biomarkers.

### Example 4. Materials and Methods

The invention was made using the following materials and methods.

### Transposome Preparation

To prepare the transposome for DNA tagmentation, the first and second partially double stranded oligonucleotides were first prepared by annealing single stranded oligonucleotides. The first partially double stranded oligonucleotide comprises the single stranded first adaptor and the first barcode and the double stranded hyperactive mosaic end sequence. The second partially double stranded oligonucleotide comprises the single stranded second adaptor and the double stranded hyperactive mosaic end sequence. Table 2 below shows the oligonucleotides used in the annealing.

TABLE 2

| | |
|---|---|
| Oligo | Volume |

First partially double stranded oligonucleotide

| | |
|---|---|
| Sense strand (100 μM): | 5 μl |
| 5′-the first adaptor-the first barcode-the sense strand | |
| of the hyperactive mosaic end sequence -3′ | |
| (the first adaptor and the first barcode do not comprise cytosine) | |
| Antisense strand (100 μM): | 5 μl |
| the antisense strand of the hyperactive mosaic end sequence | |

Second partially double stranded oligonucleotide

| | |
|---|---|
| Sense strand (100 μM): | 10 μl |
| 5′-the second adaptor-the sense strand of the hyperactive | |
| mosaic end sequence -3′ | |
| Antisense strand (100 μM): | 10 μl |
| the antisense strand of the hyperactive mosaic end sequence | |

The single stranded oligonucleotides were mixed together as shown in Table 2. The mixture was heated to 85° C. and gradually cooled down to 20° C. at −0.1° C./s rate.

The annealed partially double stranded oligonucleotides were adjusted to a final concentration of 20 μM by adding 1.5×ddH₂O (15 μl for the first partially double stranded oligonucleotide and 30 μl for the second partially double stranded oligonucleotide). Two point five microliter (2.5 μl) of each of the first and second partially double stranded oligonucleotides were mixed together and 5 μl of sterile 100% glycerol was added to the mixture. The mixture can be stored at −20° C.

To assemble the transposome and activate the transposase, two microliters (2 μl) of the mixture of the first and second partially double stranded oligonucleotides were mixed with 2 μl Tn5 transposase (available commercially) and incubated at room temperature for 30 minutes. The transposome with activated Tn5 transposase was placed on ice after the assembly.

### Tagmentation and Library Preparation

The double stranded DNA molecule comprising one or more loci of interest was tagged with the first and the second partially double stranded oligonucleotides and fragmented by incubating the DNA molecule with the transposome (4 μl) prepared above. Water was added to adjust the volume of DNA and transposome mixture to 12.5 μl and 12.5 μl of 2×TD buffer (20 mM Tris pH 7.8, 10 mM MgCl₂, 20% DMF [Dimethylformamide]) was added to bring the total reaction volume to 25 μl. Each sample was added into wells with activated transposase Tn5 (on ice) and mixed by pipetting. The reaction mix was incubated at 55° C. for 15 minutes. To stop the reaction, 6 μl of the STOP buffer (100 mM MES pH 5, 4.125M Guanidine Thiocyanate, 25% Isopropanol, 10 mM EDTA) or 0.2% SDS was added to the reaction mix and incubated at 55° C. for 7 minutes. The tagged and fragmented DNA may be pooled. The DNA sample was purified using 1.5 volumes of SPRI beads (Ampure). The DNA sample was eluted from the SPRI beads in 40 μl buffer.

To end-repair the tagged and fragmented DNA, 1.2 μg of purified DNA was diluted to 38 μl. Five microliters (5 μl) of 10×dNTPs (5-methyl-dCTP, dATP, dTTP, dGTP), 5 μl of NEB Buffer 2 (New England Biolabs), and 2 μl of Klenow (New England Biolabs) were mixed together with the DNA. The reaction mix was incubated at 37° C. for 30 minutes. One microliter (1 μl) of the end-repaired DNA may be aliquoted for quality control using TapeStation system (Agilent).

### Target Enrichment

To use in-solution target enrichment method, three mixes was prepared separately as shown in Table 3 below.

TABLE 3

| | | Hybridization | | Oligo Bait | |
|---|---|---|---|---|---|
| Library Mix | | Buffer Mix | | Library Mix | |
| | Volume | | Volume | | Volume |
| Reagent | (μl) | Reagent | (μl) | Reagent | (μl) |
| DNA | 3.4 | 20xSSPE | 25 | Nuclease | 4 |
| library | | | | free water | |
| Cot-1 DNA | 5 | 0.5M | 1 | Oligo Bait | 1 |
| (1 μg/μl) | | EDTA | | Library (500 | |
| | | | | ng/μl) | |
| Blocking | 0.6 | 50 x | 10 | RNase | 1 |
| Primers | | Denhardt's | | Inhibitor | |
| | | 1% SDS | 13 | | |
| Total | 9 | Total | 49 | Total | 6 |

The blocking primers were prepared by mixing 100 μM a first blocking primer and a second blocking primer in equal volume. The blocking primers comprises the reverse complement of the corresponding adaptor. In some embodiments, the first blocking primer has the following structure:
5′-the anti-sense strand of the hyperactive mosaic end sequence-degenerate nucleotides-the reverse complement of the first adaptor-3′

The length of the degenerate nucleotides corresponds to that of the first barcode. For example, if the first barcode is 5 nucleotides in length, the degenerate nucleotides is 5 nucleotides in length as well. An exemplary first blocking primer has the sequence 5′-CTGTCTCTTATACACATCTHHHHHCCACCCTCCACCCA-3′ (SEQ ID NO: 13). As used herein, The letter “H,” as used in sequence listing of the present invention, represents a nucleotide that is not guanine.

In some embodiments, the second blocking primer has the following structure:
5′-the anti-sense strand of the hyperactive mosaic end sequence-the reverse complement of the second adaptor-3′

An exemplary second blocking primer has the sequence 5′-CTGTCTCTTATACACATCTCCGAGCCCACGAGAC-3′ (SEQ ID NO: 14)

An exemplary second blocking primer has the sequence 5′-TCGTCGGCAGCGTCAGATGTGTATAAGAGACAG-3′ (SEQ ID NO: 20)

The concentration of the bait library was adjusted to optimize the reaction. Any appropriate methods to concentrate a DNA sample known in the art may be used. For example, the bait library may be concentrated using a speed vacuum. The Library Mix and the Oligo Bait Library Mix were kept at room temperature before use. All the reagents and equipment were nuclease free.

The hybridization was performed in a thermocycler according to the reaction profile shown in Table 4.

TABLE 4

| Step | Temperature | Time (minutes) |
|---|---|---|
| 1 | 95° C. | 5 |
| 2 | 65° C. | 3 |
| 3 | 65° C. | 2 |
| 4 | 65° C. | Forever |

At step one, the Library Mix was transferred to the thermocycler and incubated at 95° C. for five minutes on the thermocycler. At the beginning of step 2, the Hybridization Buffer Mix was transferred to the thermocycler and incubated at 65° C. for three minutes. At the beginning of step 3, the Oligo Bait Library Mix was transferred to the thermocycler and incubated at 65° C. for two minutes. At the beginning of step 4, 13 μl of the Hybridization Buffer Mix and 9 μl of the Library Mix were mixed with the 6 μl of the Oligo Bait Library Mix and mix by pipetting. The reaction mix was then incubated at 65° C. for 24 hours. The incubation time may be adjusted up to 72 hours, depending on the application.

To capture the complex formed by the hybridization of the DNA and bait oligo, streptavidin magnetic beads were used. One hundred and twenty five microliters (125 μl) of NEB streptavidin magnetic beads (New England Biolabs) was used for a capture reaction using 500 ng bait oligo. Before use, the beads were washed with 200 μl binding buffer (provided by vendor) 3 times, and then resuspend in 200 μl of binding buffer. The hybridization-capture mix was added quickly to the 200 μl beads and incubated on a rotator for 30 minutes at room temperature (20° C.). The beads were pelleted with magnetic separator and the supernatant was removed.

The beads were then resuspended in 500 μl of the first wash buffer (provided by the vendor) and incubated for 15 minutes at room temperature. The beads and the buffer were separated on a magnetic separator and the supernatant was removed. The beads was then mixed with 500 μl of the second wash buffer (provided by the vendor, pre-warmed to 65° C.) and incubated at 65° C. for 10 minutes. The second wash buffer was removed and the wash with the second wash buffer was repeated twice. The beads were then suspended in 50 μl elution buffer (freshly prepared 0.1 N NaOH from 1 N NaOH stock solution). The beads and the elution buffer were separated on a magnetic separator. The supernatant was transferred to a tube containing 70 μl of neutralization buffer. One hundred nanogram (100 ng) of lambda phage DNA was added as a carrier. The captured DNA was desalted and purified using Zymo Clean/Concentrate kit (Zymo Research). The DNA was eluted in 21 μl of buffer.

### Bisulfite Treatment

Bisulfite treatment was performed using Zymo EZ DNA Methylation-Lightning Kit according to the protocol provided by the manufacturer. The bisulfite treatment generated cytosine-converted DNA molecule.

### Amplification of the Cytosine-Converted DNA Molecules

The cytosine-converted DNA molecules were amplified using polymerase chain reaction (PCR). The PCR reaction mix was set up according to Table 5.

TABLE 5

| Reagent | volume (μl) |
|---|---|
| NEB Q5U 2X MM (New England Biolabs) | 25 |
| 25 μM Forward Primer | 1 |
| 25 μM Reverse Primer | 1 |
| Library input | 20 |
| H2O | 3 |
| Total | 50 |

The forward primer has the following structure: 5′-the first universal primer-the first sequencing primer-the first adaptor-3′. The reverse primer has the following structure: 5′-the second universal primer-the second barcode-the second adaptor-3′.

The DNA was amplified according to the cycling profile shown in Table 6.

TABLE 6

| Temp | Time | |
|---|---|---|
| 98° C. | 30 | seconds |

THEN 15 cycles of:

| | | |
|---|---|---|
| 98° C. | 10 | seconds |
| 65° C. | 30 | seconds |
| 72° C. | 1 | minutes |

HOLD at 4° C.

The annealing temperature can be adjusted empirically depending on the primer used. For example, the annealing temperature may be about 5° C. lower than the calculated Tₘ of the primers.

After 15 cycles of PCR, 10% of the reaction mix (5 μl) was aliquoted and subject to a quality control PCR to determine the number of the remaining cycles. The quality control PCR was set up according to Table 7.

TABLE 7

| Reagent | volume (μl) |
|---|---|
| H₂O | 1.5 |
| 25 μM Forward Primer | 0.5 |
| 25 μM Reverse Primer | 0.5 |
| SYBR Green 2X MM (Thermo Fisher) | 7.5 |
| Input library | 5 |

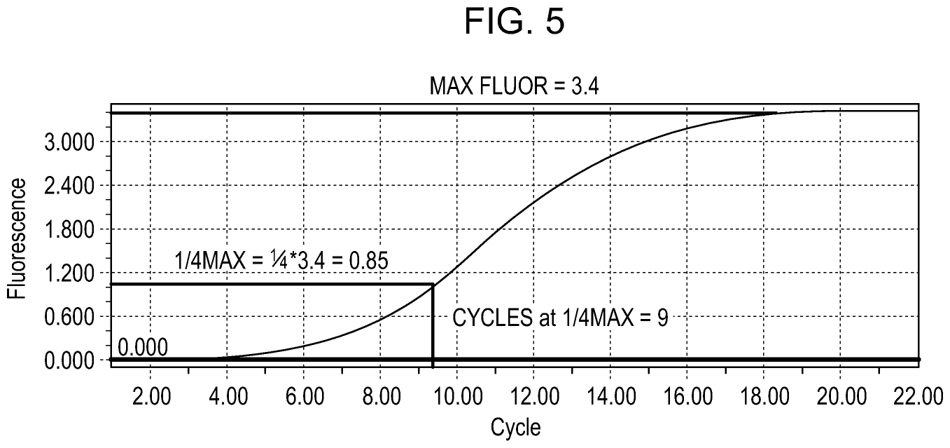

The number of remaining cycles corresponds to where the library reaches ¼ max fluorescence. FIG. 5 shows an example of the qPCR. In FIG. 5, the max fluorescence was reached at ≈3.4 FU (fluorescence unit). To reach 0.85 FU (¼×3.4), 9 additional cycles (rounding up from 8.5) were needed.

The PCR reaction that was held at 4° C. was continued with the number of cycles calculated based on the quality control qPCR using the same cycling profile. The PCR was completed with a 5 minutes extension at 72° C. The PCR product was purified with 1.8 volumes magnetic SPRI beads (Ampure) and eluted in 22 μl buffer. One microliter of the purified PCR product was aliquoted for determining the concentration using Qubit (ThermoFisher). The purified DNA was aliquoted for quality control using TapeStation.

### Example 5. Design of Tagmentation-Based Indexing for Methylation Sequencing for Targeted Methylation Sequencing and Biomarker Discovery

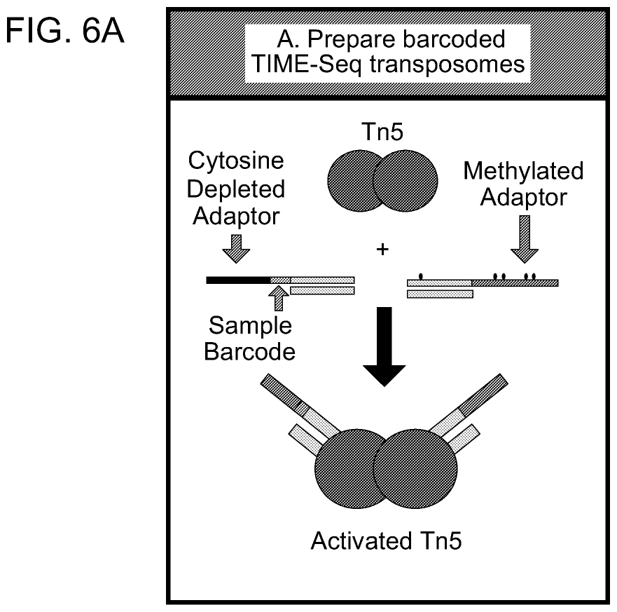

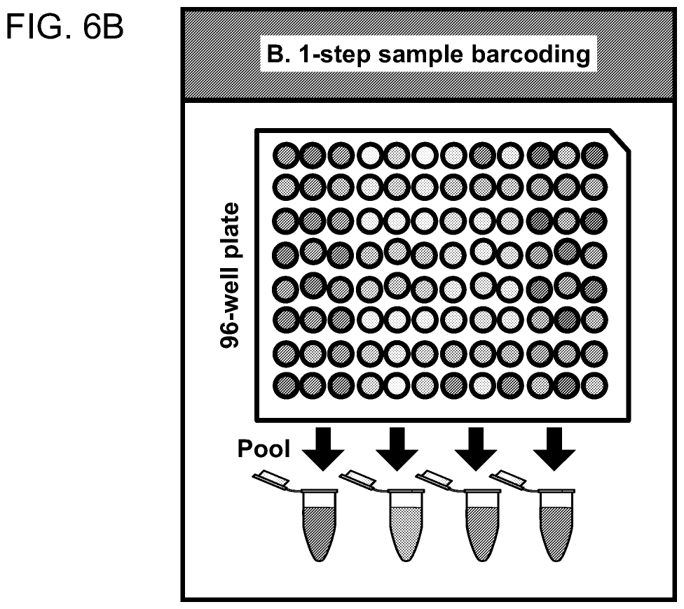

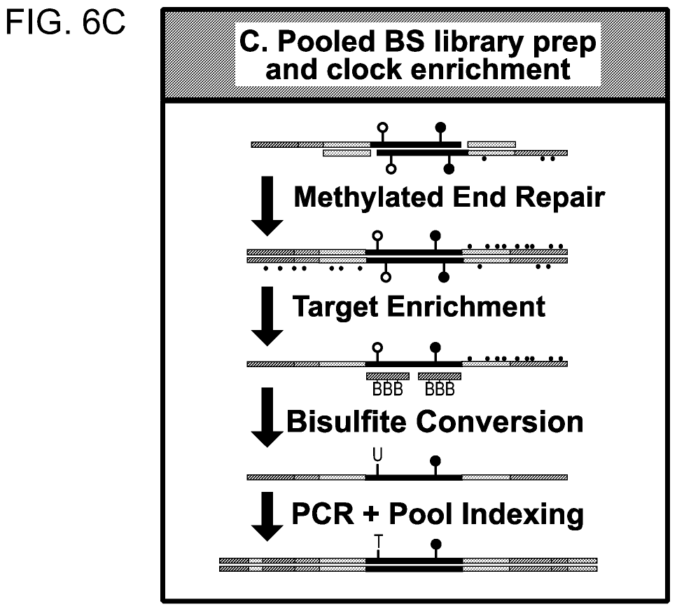

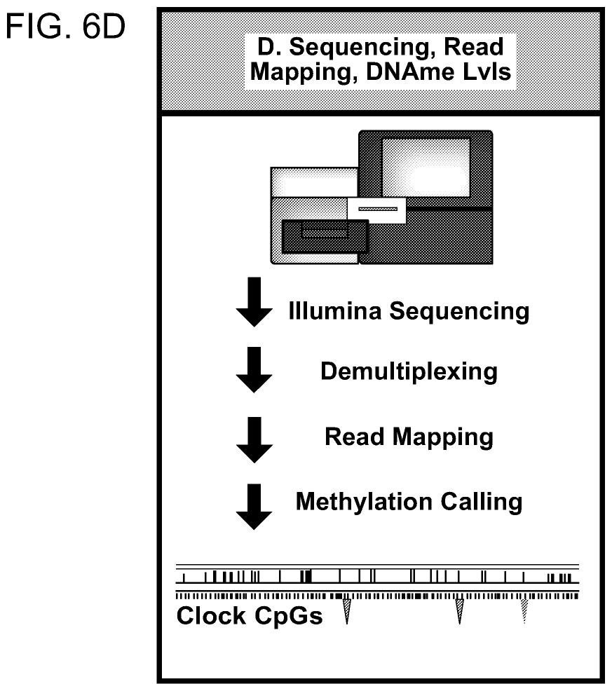

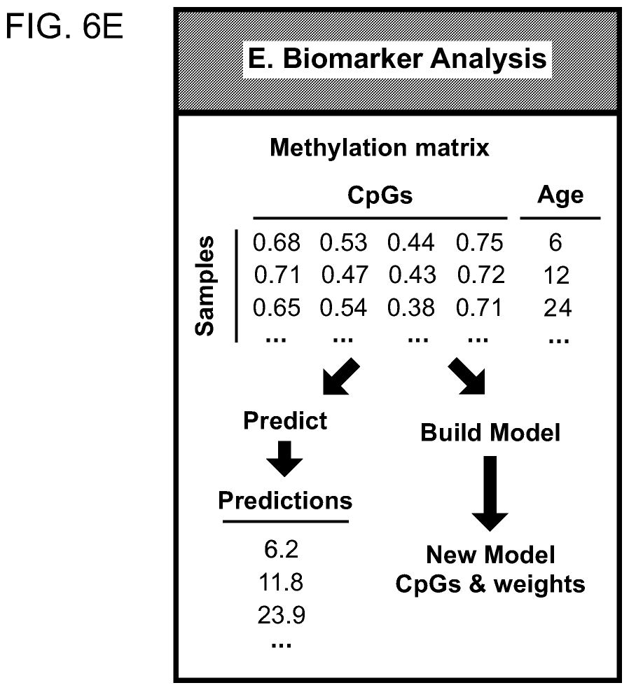

A method for rapid and inexpensive multiplexed bisulfite sequencing (BS) that is compatible with efficient hybridization enrichment, which is referred to as Tagmentation-based Indexing for MEthylation Sequencing (TIME-Seq), was designed, validated, and characterized. This method leverages the transposase Tn5 to “tagment” (fragment and tag with oligonucleotides) individual DNA samples with indexed DNA adapters (FIG. 6A). These adapters are designed to resist sequence conversion during sodium bisulfite treatment, which converts unmethylated but not methylated cytosines to uracils and is used to assay DNA methylation (DNAme) (FIG. 7A-7E). After the initial barcoding step, barcoded samples are pooled, and the library prep is completed in one tube. This drastically decreased the consumable cost of library preparation for individual samples and streamlined working with dozens or hundreds of samples. After cleaning up the pooled DNA, methylated end-repair is performed to protect the reverse strand of the adapters and DNA is enriched for targeted loci using biotinylated-RNA hybridization baits that are complementary to the regions of interest. After streptavidin pull-down of biotin-RNA:DNA complexes, pooled DNA is eluted, bisulfite converted, and amplified with pool-specific indices and the pools are combined for Illumina short-read sequencing using custom sequencing primers for index 1 and read 2. Samples are demultiplexed based on the pool and sample-specific indices, reads are mapped to the appropriate genome, and methylation is called using the software Bismark (Krueger and Andrews 2011).

### Example 6. Validation and Characterization of TIME-Seq Library Preparation and Hybridization Enrichment of Epigenetic Clock CpGs

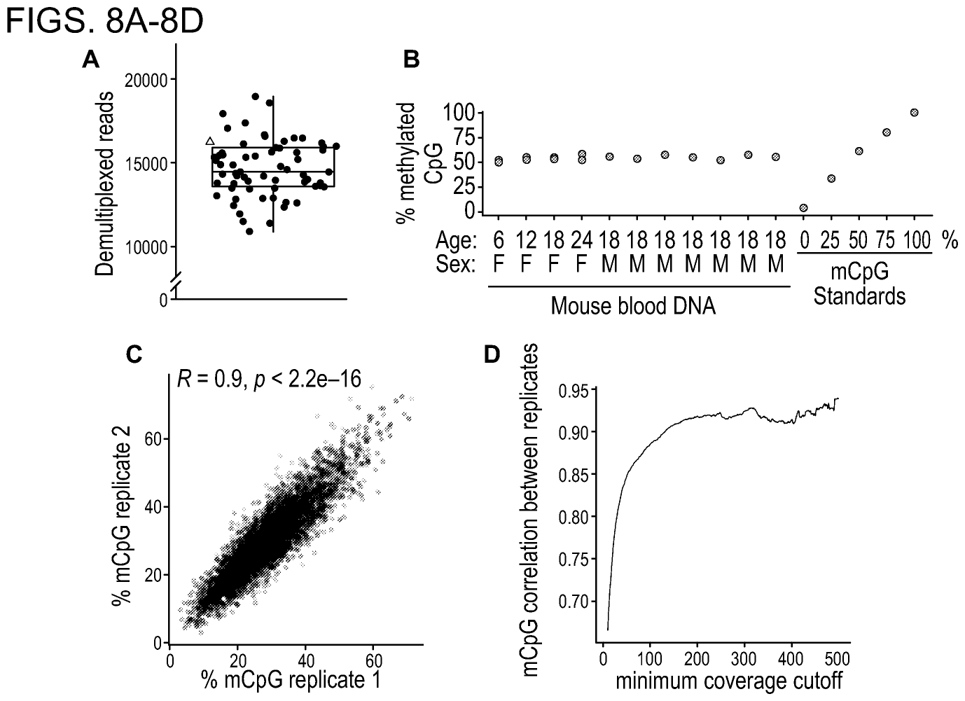

The TIME-Seq library preparation has been tested and characterized in a number of ways. To test that sample demultiplexing was possible from the internal TIME-Seq Tn5 indexes, a pool of 64 DNA samples were included in a single pool after tagmentation, the library prep was finished, the library was sequenced, and samples were demultiplexed. This experiment demonstrated that the number of reads demultiplexed from each sample is even and there are a relatively low number of unidentified reads (FIG. 8A). To test the accuracy of TIME-Seq methylation levels, libraries including mouse blood DNA from both males and females as well as methylation standards was prepared (FIG. 8B). These results demonstrate that TIME-Seq is compatible with highly accurate methylation estimation by bisulfite sequencing based on the methylation level of the standards and the expected methylation of DNA from mouse blood. To test the reproducibility of TIME-Seq libraries, two separately prepared TIME-Seq libraries were prepared from 12 mouse blood DNA samples. This data shows there is high correlation between methylation values between the replicates (FIG. 8C-8D).

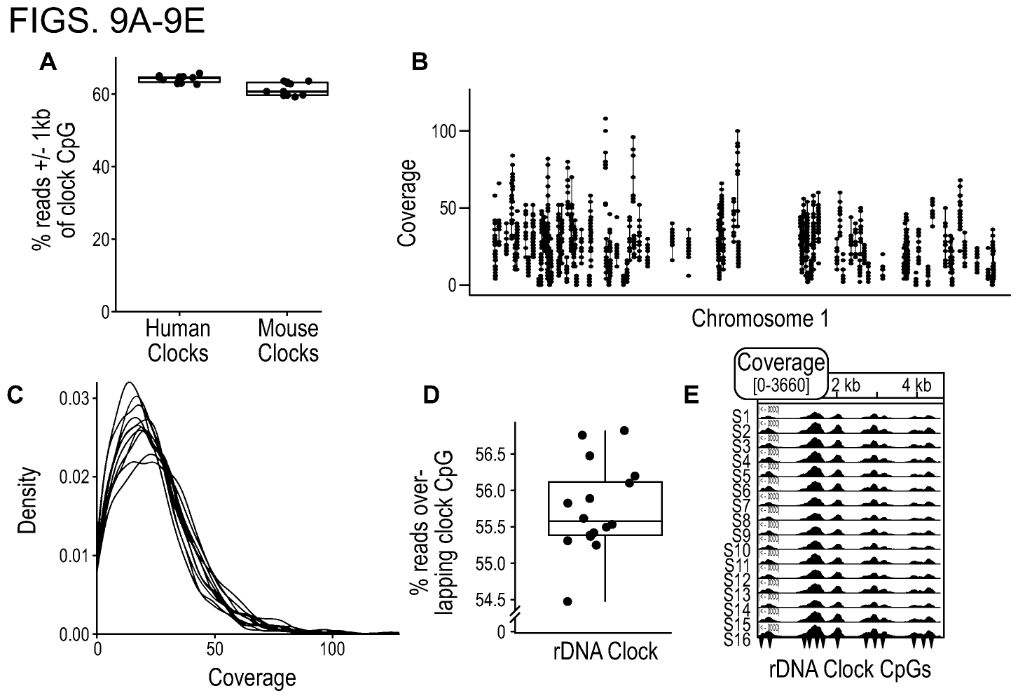

TIME-Seq adapters were specifically designed to be compatible with highly efficient hybridization-based enrichment. When using biotinylated-RNA baits that enrich for previously described human and mouse loci (Petkovich, Podolskiy et al. 2017, Liu, Leung et al. 2020), greater than 60% of sequenced reads map to within 1 kb of the target loci (FIG. 9A). Further, there is even enrichment across loci and samples within the pool (FIG. 9B-9C). When enriching for repetitive ribosomal DNA, over half the sequenced DNA mapped to ribosomal DNA (FIG. 9D), resulting in extremely high coverage at target CpGs from each sample in the TIME-Seq pool (FIG. 9E).

### Example 7. Comparison of TIME-Seq Adapters to an Alternative Adapter Design

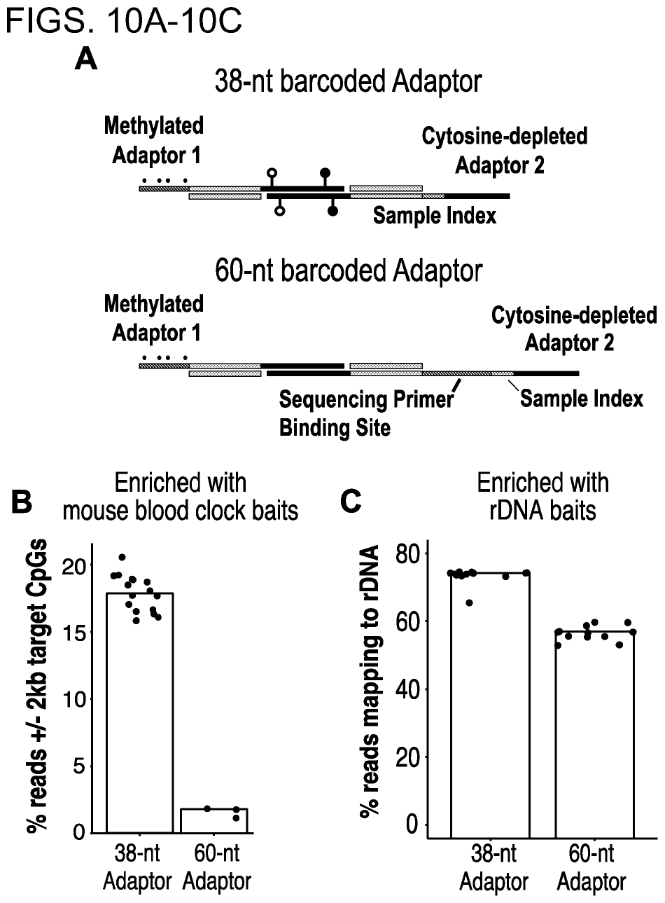

Alternative barcoded adapter designs were assessed, such as a design similar to a published single-cell methylation sequencing method (Mulqueen, Pokholok et al. 2018). This alternative approach uses longer barcoded adapters (60-nt) (FIG. 10A), and in direct comparison of the two approaches, it was found that the on-target percentage using the TIME-Seq shorter adapters was substantially higher than the alternative long adapters (FIG. 10B-10C).

More specifically, Mulqueen includes two separate adaptor regions that sandwich an index, whereas, TIME-Seq barcoded adaptor has only one adaptor region with a barcode 3′ to it. In Mulqueen, the 3′ adaptor region is a sequencing primer binding site that allows for a custom sequencing primer to bind, whereas, the second adaptor is designed to allow for PCR amplification after the addition of the second adaptor with linear amplification by random priming. One advantage of the present design is that the adaptor is shorter, which reduces daisy chaining in hybridization enrichment. In this experiment, long adapters reduced the “on-target” reads to ≈1-2% from ≈15-20% using baits targeted non-repetitive loci and also substantially reduced the enrichment of repetitive ribosomal DNA. This difference is likely due to reduced “daisy-chaining”—annealing of non-complementary off-target reads at their sequencing adapters—by the short adapters compared to the long, which has been observed in hybridization enrichment method comparisons of long and short adapters in the past (Rohland and Reich 2012).

### Example 8. Characterization of TIME-Seq Hybridization Enrichment and Library Recovery

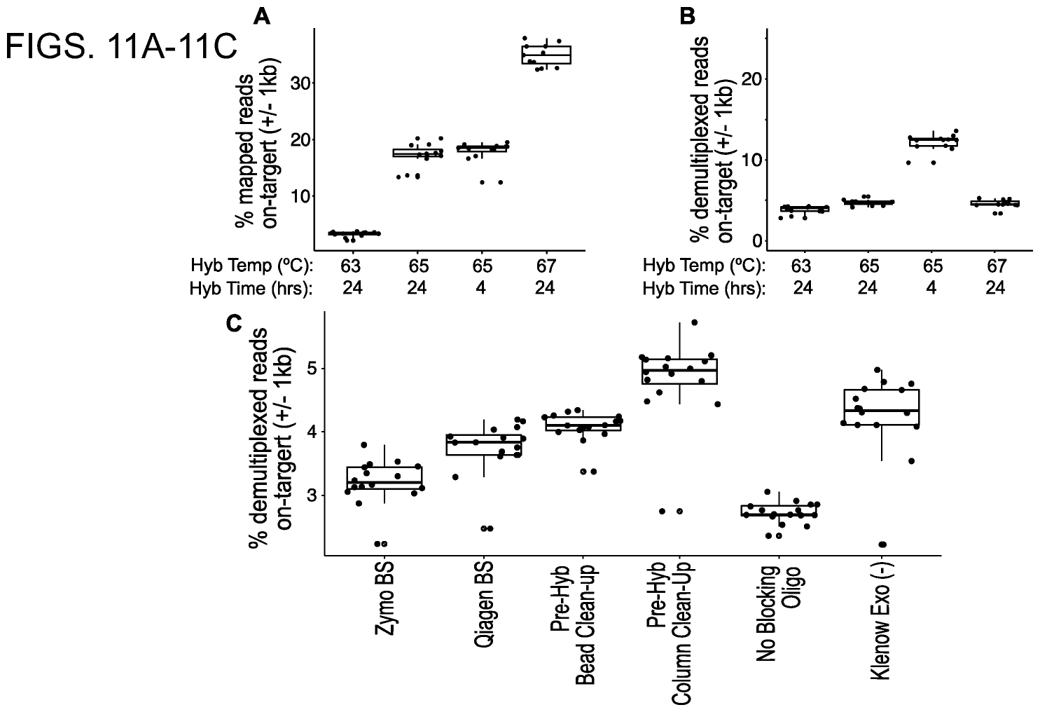

To characterize parameters of the TIME-Seq library preparation, parameters such as DNA input amount, hybridization time and temperature, pre-hybridization library clean up, end-repair, and bisulfite conversion kit were altered to identify the best conditions for the library preparation (FIG. 11A-11C). These results were incorporated into the working protocol.

### Example 9. TIME-Seq Enables Inexpensive and Highly Accurate Estimation of Age in Mice Based on Ribosomal DNA Methylation

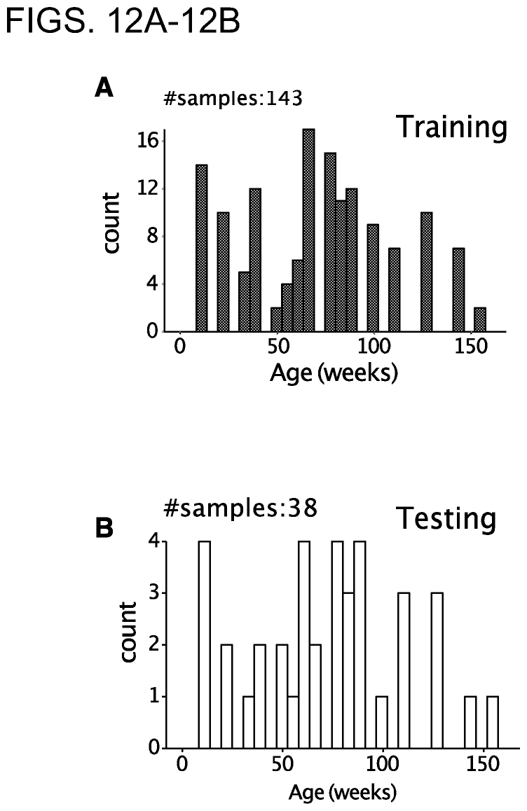

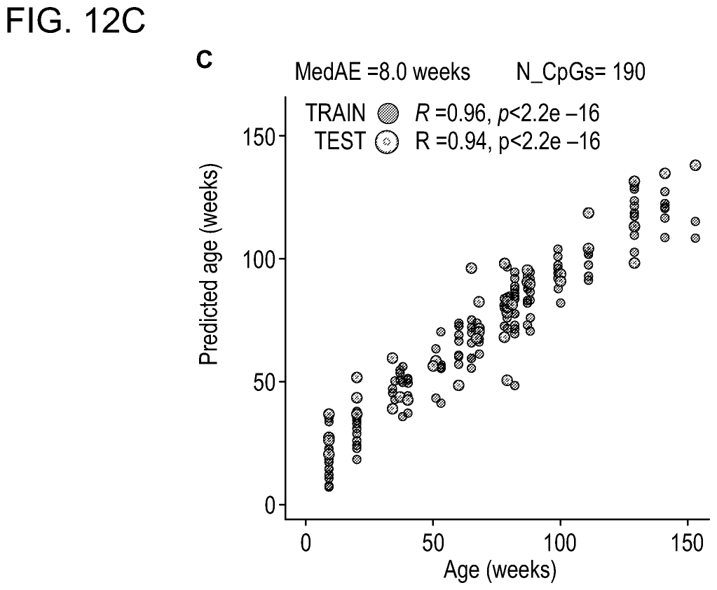

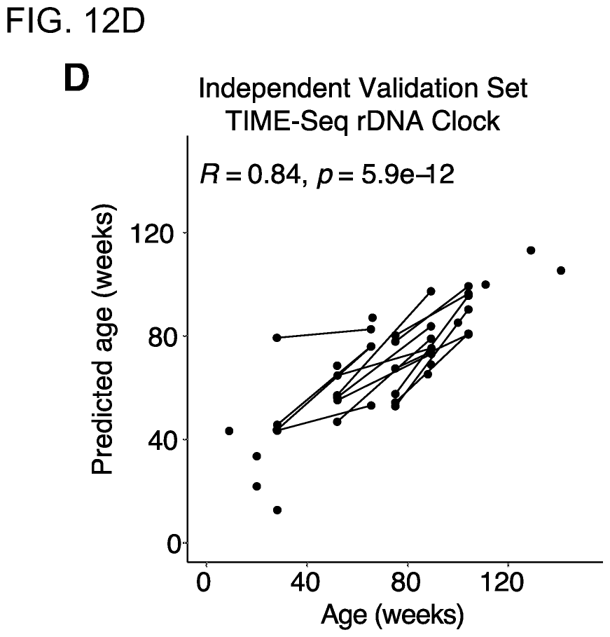

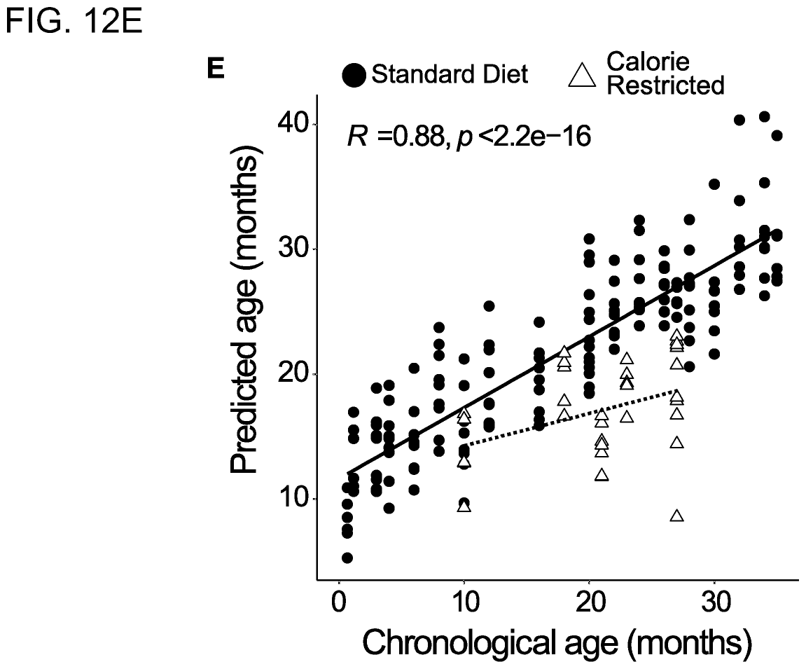

To build a TIME-Seq specific DNA methylation clock, libraries from 181 mouse blood samples and enriched for ribosomal DNA (rDNA), which has been shown to be ideal for epigenetic clock analysis (Wang and Lemos 2019), were prepared. Pools were sequenced using an Illumina MiSeq and the per sample sequence cost was approximately $5. After mapping the reads to a representative rDNA locus, samples were split into training and testing sets (FIG. 12A-12B) and elastic net regression was applied to identify a CpGs and weights comprising a model for a DNA methylation clock. Both training (R=0.96) and testing (R=0.94) sets had extremely high correlation with age and a median absolute error in estimation of only two months (FIG. 12C). To validate the clock, a separate TIME-Seq library of 40 sample, some which had longitudinal time points, was prepared (FIG. 12D). This experiment demonstrated that the clock is accurate in an independent cohort and reflects longitudinal age change. A clock from TIME-Seq data that only used CpGs common to RRBS data was also built and this clock was applied to a previously described RRBS dataset (Petkovich, Podolskiy et al. 2017). This showed that the TIME-Seq clocks could accurately predict age in RRBS samples (R=0.88) and reflected life-extending caloric restriction intervention (FIG. 12E).

### Example 10. Highly Accurate Age Prediction from Shallow Sequencing of TIME-Seq Libraries

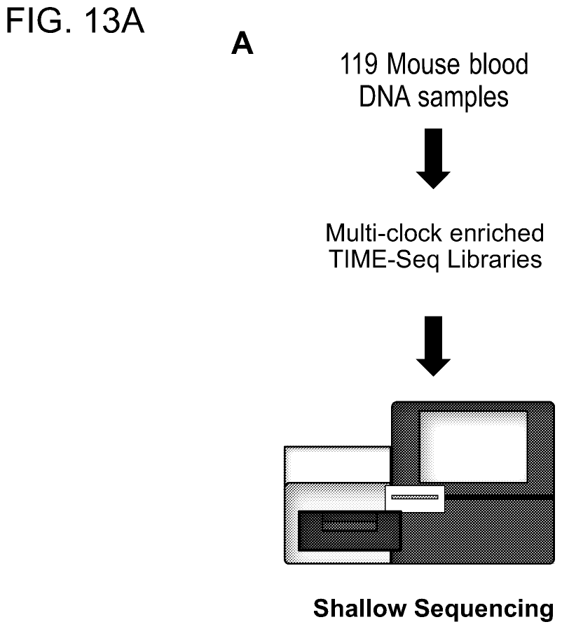

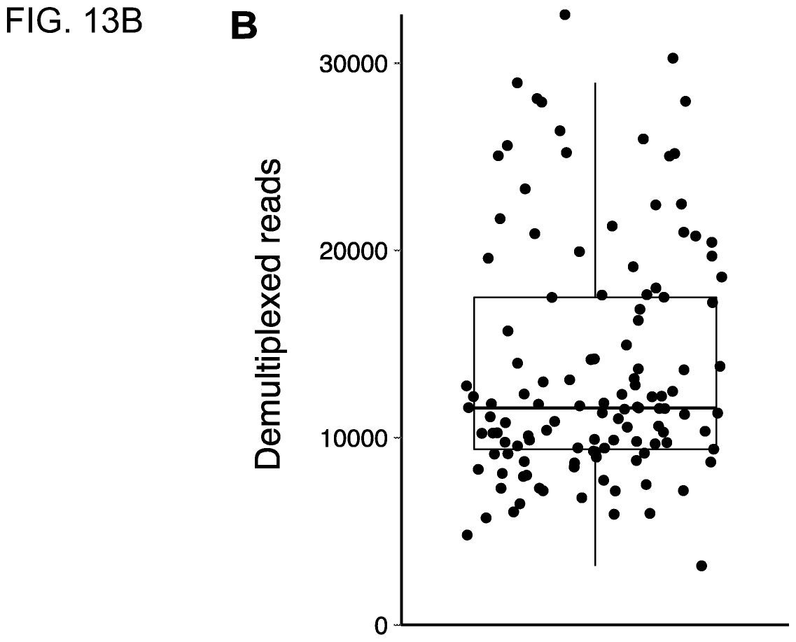

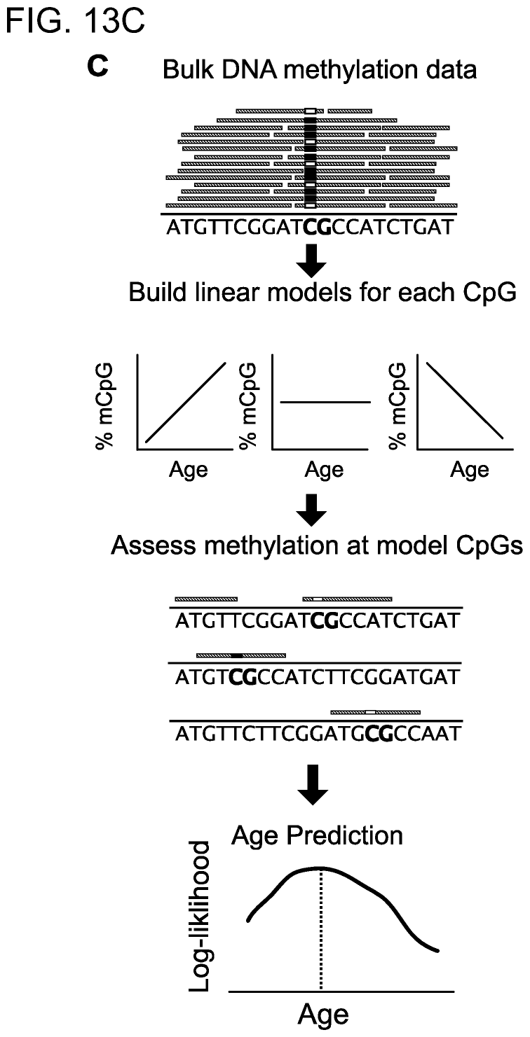

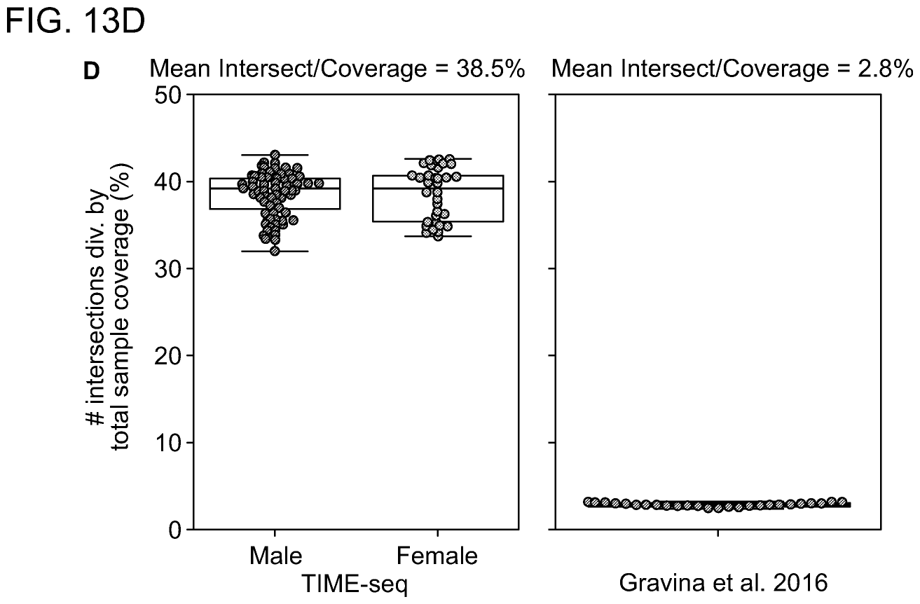

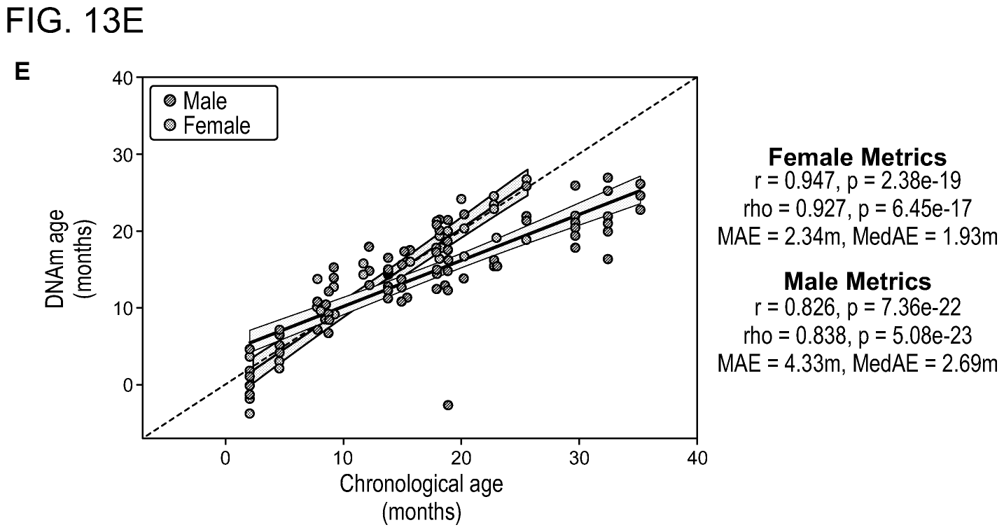

In view of recent work predicting age in single cells using an algorithm called scAge (Trapp, Kerepesi et al. 2021), this experiment tests whether it might be possible to predict age from shallow sequencing of TIME-Seq libraries (FIG. 13A). These libraries were only sequenced with 5 to 30 thousand reads per sample (FIG. 13B), creating a sparse methylation matrix in which most CpGs were covered by only 1-2 reads and most samples had very few overlapping CpGs in a pairwise comparison (data not shown). To predict age, a modified version of the scAge algorithm was applied to the TIME-Seq data (FIG. 13C). The algorithm works by creating linear models to predict methylation from age using a previously described bulk sequenced dataset and then taking a maximum likelihood approach to predict age from the shallow sequencing data. Due to the targeted nature of TIME-Seq libraries, it is especially amenable to this approach for age prediction since the CpGs that are covered in TIME-Seq libraries are highly overlapping with those included in the linear models (median 38.5%) when compared to non-enriched random DNA methylation sequencing such as you would see in single cell methylation data (median 2.8%) (FIG. 13D). Surprisingly, age predictions from shallow sequencing of TIME-Seq data were incredibly accurate, with correlations of R=0.947 in females and R=0.826 in males, with median absolute error of prediction 1.93 and 2.69 months respectively. Given the per sample sequencing cost of less than $2 per sample, this is the most cost-effective epigenetic clock measurement in a large cohort of mice or humans to date. These results were surprising at least because the results were as accurate as many deeply sequenced clocks (i.e., coverage of 50-100+ reads per CpG) and this was the first demonstration that age prediction from shallow sequencing (i.e., coverage of 1-2 reads per CpG) could be as accurate as from bulk sequencing, using this method or any other.

### BIBLIOGRAPHY

Gravina, S., X. Dong, B. Yu and J. Vijg (2016). “Single-cell genome-wide bisulfite sequencing uncovers extensive heterogeneity in the mouse liver methylome.” Genome Biol 17(1): 150.
Krueger, F. and S. R. Andrews (2011). “Bismark: a flexible aligner and methylation caller for Bisulfite-Seq applications.” Bioinformatics 27(11): 1571-1572.
Liu, Z., D. Leung, K. Thrush, W. Zhao, S. Ratliff, T. Tanaka, L. L. Schmitz, J. A. Smith, L. Ferrucci and M. E. Levine (2020). “Underlying features of epigenetic aging clocks in vivo and in vitro.” Aging Cell: e13229.
Mulqueen, R. M., D. Pokholok, S. J. Norberg, K. A. Torkenczy, A. J. Fields, D. Sun, J. R. Sinnamon, J. Shendure, C. Trapnell, B. J. O'Roak, Z. Xia, F. J. Steemers and A. C. Adey (2018). “Highly scalable generation of DNA methylation profiles in single cells.” Nat Biotechnol 36(5): 428-431.
Petkovich, D. A., D. I. Podolskiy, A. V. Lobanov, S. G. Lee, R. A. Miller and V. N. Gladyshev (2017). “Using DNA Methylation Profiling to Evaluate Biological Age and Longevity Interventions.” Cell Metab 25(4): 954-960.e956.
Rohland, N. and D. Reich (2012). “Cost-effective, high-throughput DNA sequencing libraries for multiplexed target capture.” Genome Res 22(5): 939-946.
Trapp, A., C. Kerepesi and V. N. Gladyshev (2021). “Profiling epigenetic age in single cells.” bioRxiv: 2021.2003.2013.435247.
Wang, M. and B. Lemos (2019). “Ribosomal DNA harbors an evolutionarily conserved clock of biological aging.” Genome Res.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present invention described herein. Such equivalents are intended to be encompassed by the following claims.

### INFORMAL SEQUENCE LISTING

Table 8 below shows the SEQ ID NOs of the exemplary oligonucleotides described herein.

TABLE 8

| SEQ | | |
|---|---|---|
| ID | | |
| NO | Description | Sequence |
| 1 | ME Sense strand | AGA TGT GTA TAA GAG ACA G |
| 2 | ME antisense strand | C TGT CTC TTA TAC ACA TCT |
| 3 | Cytosine free oligo | DDD DDD DDD DDD DDD |
| 4 | Adaptor A | TGG GTG GAG GGT GG |
| 5 | Adaptor B | GTmC TmCG TGG GmCT mCGG |
| 16 | Adaptor B | TmCG TmCG GmCA GmCG TmC |
| 6 | Adaptor A-Barcode- | TGGGTGGAGGGTGGDDDDDAGATGT |
| | ME | GTATAAGAGACAG |
| 21 | Adaptor B-ME | GTmCTmCGTGGGmCTmCGGAGATGT |
| | | GTATAAGAGACAG |
| 7 | Adaptor B-ME | GTmCTmCGTGGGmCTmCGGAGATGT |
| | | GTATAAGAGAmCAG |
| 17 | Adaptor B-ME | TmCGTmCGGmCAGmCGTmCAGATGT |
| | | GTATAAGAGACAG |
| 8 | Forward Primer | AATGATACGGCGACCACCGAAAGCAGTGGTATCAAC |
| | | GCAGATCTGGGTGGAGGGTGGTGGGTGGAGGGTGG |
| 18 | Forward Primer | AATGATACGGCGACCACCGAGATCTACACNNNNNNN |
| | | NTCGTCGGCAGCGTC |
| 9 | Reverse Primer | CAAGCAGAAGACGGCATACGAGATNNNNNNNNGTCT |
| | | CGTGGGCTCGGAGATGT |
| 19 | Reverse Primer | CAAGCAGAAGACGGCATACGAGATNNNNNNNNAAGC |
| | | AGTGGTATCAACGCAGATCTGGGTGGAGGGTGG |
| 10 | Illumina P5 | AAT GAT ACG GCG ACC ACC GA |
| 11 | Illumina P7 | CAA GCA GAA GAC GGC ATA CGA GAT |
| 12 | First Sequencing | AAG CAG TGG TAT CAA CGC |
| | Primer | AGA TCT GGG TGG AGG |
| | | GTG G |
| 13 | First blocking | CTGTCTCTTATACACATCTHHHHHCCACCCTCC |
| | primer | ACCCA |
| 14 | Second blocking | CTGTCTCTTATACACATCTCCGAGCCCACGAGAC |
| | primer | |
| 20 | Second blocking | TCGTCGGCAGCGTCAGATGTGTATAAGAGACAG |
| | primer | |
| 15 | Second Custom | CCACCCTCCACCCAGATCTGCGTTGATACCAC |
| | Sequencing Primer | TGCTT |
| | (Index i7) | |

What is claimed is:

1. A method for assembling an enzyme-deoxyribonucleic acid (DNA) complex for use in preparing a double stranded DNA molecule comprising one or more loci of interest for determining the methylation status of the one or more loci of interest therein, comprising:
contacting an enzyme with a first partially double stranded oligonucleotide comprising a first adaptor single stranded oligonucleotide and a first barcode single stranded oligonucleotide, wherein the first adaptor oligonucleotide and the first barcode oligonucleotide are operably linked in the order, from 5′ to 3′, the first adaptor-the first barcode, and
a second partially double stranded oligonucleotide comprising a second adaptor single stranded oligonucleotide,
wherein the enzyme is capable of operably linking the first and the second partially double stranded oligonucleotides to the double stranded DNA molecule comprising one or more loci of interest;
wherein the first adaptor and the first barcode do not comprise a cytosine,
wherein the second adaptor does not comprise a cytosine or the cytosine thereon is methylated; and
wherein the nucleotide sequence of the first adaptor and the second adaptor are different,
thereby preparing the enzyme-DNA complex.

2. The method of claim 1, wherein the first partially double stranded oligonucleotide further comprises a first enzyme recognition sequence, wherein the first enzyme recognition sequence is operably linked to the 3′-terminus of the first barcode; and
wherein the second partially double stranded oligonucleotide further comprises a second enzyme recognition sequence, wherein the second enzyme recognition sequence is operably linked to the 3′-terminus of the second adaptor.

3. The method of claim 2, wherein the first enzyme recognition sequence is a first transposon end sequence for a transposon, and wherein the second enzyme recognition sequence is a second transposon end sequence for the transposon.

4. The method of claim 1, wherein the enzyme is a transposase, and the enzyme-DNA complex is a transposome.

5. The method of claim 4, wherein the transposon is transposon 5 (Tn5).

6. The method of claim 1, wherein the enzyme is a hyperactive transposase Tn5.

7. The method of claim 3, wherein the transposon end sequence comprises a hyperactive mosaic end (ME) nucleotide sequence.

8. The method of claim 7, wherein the nucleotide sequence of the sense strand of the ME sequence is at least about 85% identical to the entire nucleotide sequence of a nucleotide sequence having the sequence of SEQ ID NO: 1.

9. The method of any one of claims 1-8, wherein the first adaptor is between 6 nucleotides and 30 nucleotides in length.

10. The method of claim 9, wherein the first adaptor 14 nucleotides in length.

11. The method of claim 9 or 10, wherein the first adaptor comprises a nucleotide sequence having at least about 85% nucleotide identity to the entire nucleotide sequence of SEQ ID NO: 4, wherein the first adaptor does not comprise a cytosine.

12. The method of any one of claims 1-11, wherein the second adaptor is between 6 nucleotides and 30 nucleotides in length.

13. The method of claim 12, wherein the second adaptor is 15 nucleotides in length.

14. The method of claim 12 or 13, wherein the second adaptor comprises a nucleotide sequence having at least about 85% nucleotide identity to the entire nucleotide sequence of SEQ ID NO: 5, wherein the cytosine on the second adaptor is methylated.

15. The method of claim 14, wherein the second adaptor comprises a nucleotide sequence having entire nucleotide sequence of SEQ ID NO: 5.

16. The method of claim 12 or 13, wherein the second adaptor comprises a nucleotide sequence having at least about 85% nucleotide identity to the entire nucleotide sequence of SEQ ID NO: 16, wherein the cytosine on the second adaptor is methylated.

17. The method of claim 16, wherein the second adaptor comprises a nucleotide sequence having entire nucleotide sequence of SEQ ID NO: 16.

18. The method of any one of claims 1-17, wherein the first barcode comprises a nucleotide sequence selected from the group consisting of DDDDD and DDDDDD.

19. A method of preparing a double stranded deoxyribonucleic acid (DNA) molecule comprising one or more loci of interest for determining the methylation status of one or more loci of interest therein, comprising:
providing a double stranded DNA molecule comprising one or more loci of interest,
contacting the double stranded DNA molecule comprising one or more loci of interest with the enzyme-DNA complex prepared according to the method of any one of claims 1-18.

20. A method of preparing a double stranded deoxyribonucleic acid (DNA) molecule comprising one or more loci of interest for determining the methylation status of the one or more loci of interest therein, comprising:
providing a double stranded DNA molecule comprising one or more loci of interest, the DNA molecule comprising a first strand and a second strand;
operably linking a first partially double stranded oligonucleotide comprising a first adaptor single stranded oligonucleotide and a first barcode single stranded oligonucleotide to the 5′-terminus of the first strand of the double stranded DNA molecule in the order, from 5′ to 3′, the first adaptor-the first barcode-the double strand DNA molecule; and
operably linking a second partially double stranded oligonucleotide comprising a second adaptor single stranded oligonucleotide to the 5′-terminus of the second strand of the DNA molecule,
wherein the first adaptor and the first barcode do not comprise a cytosine,
wherein the second adaptor does not comprise a cytosine or the cytosine thereon is methylated; and
wherein the nucleotide sequence of the first adaptor and the second adaptor are different,
thereby preparing the double stranded DNA comprising one or more loci of interest for determining the methylation status of the one or more loci of interest therein.

21. The method of claim 20, wherein the first partially double stranded oligonucleotide further comprises a first enzyme recognition sequence, wherein the first enzyme recognition sequence is operably linked to the 3′-terminus of the first barcode and the 5′-terminus of the first strand of the DNA; and
wherein the second partially double stranded oligonucleotide further comprises a second enzyme recognition sequence, wherein the second enzyme recognition sequence is operably linked to the 3′-terminus of the second adaptor and the 5′-terminus of the second strand of the DNA.

22. The method of claim 21, wherein the first enzyme recognition sequence is a first end sequence for a transposon, and wherein the second enzyme recognition sequence is a second end sequence for the transposon.

23. The method of claim 22, wherein the transposon is a hyperactive transposon 5 (Tn5).

24. The method of claim 22 or 23, wherein the end sequence comprises a hyperactive mosaic end (ME) nucleotide sequence.

25. The method of claim 24, wherein the nucleotide sequence of the sense strand of the ME sequence is at least about 85% identical to the entire nucleotide sequence of SEQ ID NO: 1.

26. The method of any one of claims 20-25, further comprising assembling a transposome, comprising contacting a transposase with the first partially double stranded oligonucleotide and the second partially double stranded oligonucleotide.

27. The method of claim 26, further comprising contacting the transposome with the double stranded DNA molecule comprising one or more loci of interest, wherein the transposome fragments the double stranded DNA molecule comprising one or more loci of interest and operably links the first partially double stranded oligonucleotide and the second partially double stranded oligonucleotide to the double stranded DNA molecule comprising one or more loci of interest.

28. The method of any one of claims 20-27, wherein the first adaptor is between 6 nucleotides and 30 nucleotides, or between 14 nucleotides and 20 nucleotides in length.

29. The method of claim 28, wherein the first adaptor is 14 nucleotides in length.

30. The method of claim 28 or 29, wherein the first adaptor comprises a nucleotide sequence having at least about 85% nucleotide identity to the entire nucleotide sequence of SEQ ID NO: 4, wherein the first adaptor does not comprise a cytosine.

31. The method of any one of claims 20-30, wherein the second adaptor is between 6 nucleotides and 30 nucleotides in length.

32. The method of claim 31, wherein the second adaptor is 15 nucleotides in length.

33. The method of claim 30 or 31, wherein the second adaptor comprises a nucleotide sequence having at least about 85% nucleotide identity to the entire nucleotide sequence of SEQ ID NO: 5, wherein the cytosine on the second adaptor is methylated.

34. The method of claim 28 or 29, wherein the second adaptor comprises a nucleotide sequence having at least about 85% nucleotide identity to the entire nucleotide sequence of SEQ ID NO: 16, wherein the cytosine on the second adaptor is methylated.

35. The method of any one of claims 20-34, wherein the first barcode comprises a nucleotide sequence selected from the group consisting of DDDDD and DDDDDD.

36. The method of any one of claims 20-35, further comprising repairing the ends of double stranded DNA molecule comprising one or more loci of interest operably linked to the first partially double stranded oligonucleotide and the second partially double stranded oligonucleotide using methylated cytosine, thereby generating an end repaired double stranded DNA comprising one or more loci of interest.

37. The method of claim 36, wherein a Klenow, a T4 polymerase, or a mixture thereof is used for the end repairing.

38. The method of claim 36 or 37, further comprising enriching the DNA molecule comprising one or more loci of interest following end repairing, thereby generating a enriched DNA molecule comprising one or more loci of interest.

39. The method of claim 38, wherein the enrichment method is an in-solution target enrichment method.

40. The method of claim 39, wherein the enrichment comprises in-solution biotinylated RNA bait hybridization.

41. The method of any one of claims 36-40, further comprising converting the unmethylated cytosine in the end repaired double stranded DNA molecule comprising one or more loci of interest or the enriched DNA molecule comprising one or more loci of interest to uracil, thereby generating a cytosine-converted DNA molecule comprising one or more loci of interest.

42. The method of claim 41, wherein the unmethylated cytosine is converted into uracil via bisulfite treatment.

43. The method of claim 41 or 42, further comprising amplifying the cytosine-converted DNA molecule comprising one or more loci of interest, thereby generating an amplified double stranded DNA molecule comprising one or more loci of interest.

44. The method of claim 43, wherein the amplification comprises polymerase chain reaction (PCR).

45. The method of claim 43 or 44, further comprising operably linking a double stranded oligonucleotide comprising a first universal primer and a first sequencing primer to the first adaptor and a second double stranded oligonucleotide comprising a second universal primer and a second barcode to the second adaptor, wherein the nucleotide sequence of the first universal primer and the second universal primer is different.

46. The method of claim 45, wherein the cytosine converted DNA molecule comprising one or more loci of interest, the first universal primer, and the first sequencing primer are operably linked in the followed order: 5′-the first universal primer-the first sequencing primer-the cytosine converted DNA-3′.

47. The method of claim 46, wherein the first universal primer comprises a nucleotide sequence having about at least 85% nucleotide identity to the entire nucleotide sequence of SEQ ID NO: 10.

48. The method of claim 46 or 47, wherein the first sequencing primer is between 15 base pair to 30 base pair in length.

49. The method of claim 48, wherein the first sequencing primer comprises a nucleotide sequence having about at least 85% nucleotide identity to the entire nucleotide sequence of SEQ ID NO: 12.

50. The method of any one of claims 45-49, wherein the cytosine converted DNA molecule comprising one or more loci of interest, the second universal primer, and the second barcode are operably linked in the following order: 5′-the second universal primer-the second barcode-the cytosine converted DNA.

51. The method of claim 50, wherein the second universal primer comprises a nucleotide sequence having about at least 85% nucleotide identity to the entire nucleotide sequence of SEQ ID NOs: 11.

52. The method of claim 50 or 51, wherein the second barcode is between 6 nucleotides and 15 nucleotides in length.

53. The method of claim 52, wherein the second barcode has a length of 8 nt.

54. The method of any one of claims 45-53, wherein the first double stranded oligonucleotide and the second double stranded oligonucleotide are operably linked to the cytosine-converted DNA by PCR.

55. A method for determining the methylation status of a loci of interest, comprising preparing an amplified double stranded DNA molecule comprising one or more loci of interest according to the method of any one of the claims 43-54 and sequencing the double stranded DNA molecule, thereby determining the methylation status of the loci of interest.

56. A method for constructing a sequencing library for determining the methylation status of one or more loci of interest, comprising:
(a) fragmenting genomic DNA comprising one or more loci of interest to generate a plurality of double strand DNA molecules, wherein at least one of the plurality of double stranded DNA molecules comprises the one or more loci of interest; and
(b) preparing the plurality of double stranded DNA molecules comprising the one or more loci of interest according to the method of any one of claims 43-54,
thereby generating a sequencing library for determining the methylation status of one or more loci of interest.

57. The method of claim 56, wherein the genomic DNA is human genomic DNA.

58. A method of determining the methylation status of one or more loci of interest, comprising:
(a) preparing a sequencing library according to the method of claim 53; and
(b) sequencing the one or more loci of interest;
thereby determining the methylation status of one or more loci of interest.

59. A method of determining the methylation status of one or more loci of interest present in a plurality of subject samples, comprising
(a) constructing a sequencing library from each subject sample according to the method of any one of claims 56-58, wherein each library comprises a plurality of the double stranded DNA molecules comprising one or more loci of interest and wherein each of the first barcodes in each of the plurality of libraries is a unique first bar code;
(b) pooling the plurality of libraries; and
(c) sequencing the plurality of double stranded DNA molecules comprising one or more loci of interest;
thereby determining the methylation status of one or more loci of interest present in the plurality of subject samples.

60. The method of claim 59, wherein each of the second barcodes in each of the plurality of libraries is a unique second bar code

61. The method of any one of claims 55, 56, and 58-60, further comprising comparing the methylation status of one or more loci of interest to a reference methylation status.

62. The method of claim 61, wherein the comparison comprises comparison of the number of nucleotides comprising a methylated cytosine, the location of the methylated cytosine, or both.

63. A kit for preparing a double stranded DNA molecule comprising one or more loci of interest for determining the methylation status of the one or more loci of interest therein, comprising:
a first partially double stranded oligonucleotide comprising a first adaptor single stranded oligonucleotide and a first barcode single stranded oligonucleotide; and
a second partially double stranded oligonucleotide comprising second adaptor;
wherein the first adaptor and the nucleotide sequence of the first barcode do not comprise a cytosine,
wherein the second adaptor does not comprise a cytosine or the cytosine thereon is methylated; and
wherein the first adaptor and the first barcode are operably linked, from 5′-terminus to 3′-terminus in the following order, the first adaptor-the first barcode.

64. The kit of claim 63, wherein the first partially double stranded oligonucleotide further comprises a first enzyme recognition sequence, wherein the first enzyme recognition sequence is operably linked to the 3′-terminus of the first barcode; and
wherein the second partially double stranded oligonucleotide further comprises a second enzyme recognition sequence, wherein the second enzyme recognition sequence is operably linked to the 3′-terminus of the second adaptor.

65. The kit of claim 64, wherein the first enzyme recognition sequence and the second enzyme recognition sequence are specific site that an enzyme recognizes, and wherein the enzyme catalyzes the insertion of the first partially double stranded DNA and the second partially double stranded DNA to the 5′-terminus and 3′-terminus of a double stranded DNA molecule, respectively.

66. The kit of claim 65, further comprising the enzyme.

67. The kit of claim 65 or 66, wherein the first enzyme recognition sequence is a first end sequence for a transposon, wherein the second enzyme recognition sequence is a second end sequence for the transposon, and wherein the enzyme is a transposase.

68. The kit of claim 67, wherein the transposon is transposon 5 (Tn5) and the transposase is a hyperactive transposase Tn5.

69. The kit of claim 67 or 68, wherein the end sequence comprises a hyperactive mosaic end (ME) nucleotide sequence.

70. The kit of claim 63, wherein the first partially double stranded oligonucleotide further comprises a first barcode.

71. A method of predicting age in a plurality of subject samples, comprising
(a) constructing a sequencing library from each subject sample according to the method of any one of claims 56-58, wherein each library comprises a plurality of the double stranded DNA molecules comprising one or more loci of interest and wherein each of the first barcodes in each of the plurality of libraries is a unique first bar code;
(b) pooling the plurality of libraries;
(c) shallow sequencing the plurality of double stranded DNA molecules comprising one or more loci of interest;
(d) applying an algorithm to create a linear model to predict methylation from age using a previously described bulk sequenced dataset;
(e) taking a maximum likelihood approach to predict age from the shallow sequencing data;
thereby determining the age in the plurality of subject samples.

72. The method of claim 71, wherein each of the second barcodes in each of the plurality of libraries is a unique second bar code

73. The method of any claim 71 or 72, further comprising comparing the methylation status of one or more loci of interest to a reference methylation status.

74. The method of claim 73, wherein the comparison comprises comparison of the number of nucleotides comprising a methylated cytosine, the location of the methylated cytosine, or both.

75. The method of claim 71, wherein the shallow sequencing comprises coverage of about 1 to about 2 reads per CpG.

76. The method of claim 71, wherein the algorithm is a scAge algorithm or a modified version thereof.
